(19) United States
(12) Patent Application Publication
(10) Pub. No.: US 2021/0228754 A1
(43) Pub. Date: Jul. 29, 2021

(54) COMPOSITION, SPRAY, AND WIPER

(71) Applicant: FUJIFILM Corporation, Tokyo (JP)

(72) Inventors: Miyoko HARA, Minami-ashigara-shi (JP); Naotoshi SATO, Minami-ashigara-shi (JP); Hiroki SUGIURA, Minami-ashigara-shi (JP); Yusuke KURIMOTO, Minami-ashigara-shi (JP); Ayaka TOMISE, Minami-ashigara-shi (JP)

(73) Assignee: FUJIFILM Corporation, Tokyo (JP)

(21) Appl. No.: 17/231,578

(22) Filed: Apr. 15, 2021

Related U.S. Application Data
(63) Continuation of application No. PCT/JP2019/041318, filed on Oct. 21, 2019.

(30) Foreign Application Priority Data
Oct. 31, 2018 (JP) 2018-205851

(51) Int. Cl. A61L 2/22; A61L 2/232

(52) U.S. Cl. CPC A61L 2/22; A61L 2101/36; A61L 2/232

(57) ABSTRACT

An object of the present invention is to provide a composition having excellent antiviral activity. Another object of the present invention is to provide a spray and a wiper that use the above composition.

An alkaline composition according to an embodiment of the present invention contains a compound having an amino group and at least one kind of functional group selected from the group consisting of an acidic group, a hydroxyl group, and a phenyl group and having a molecular weight equal to or higher than 85, and a solvent containing an alcohol.

### CROSS-REFERENCE TO RELATED APPLICATIONS

This application is a Continuation of PCT International Application No. PCT/JP2019/041318 filed on Oct. 21, 2019, which claims priority under 35 U.S.C. § 119(a) to Japanese Patent Application No. 2018-205851 filed on Oct. 31, 2018. The above application is hereby expressly incorporated by reference, in its entirety, into the present application.

### BACKGROUND OF THE INVENTION

### 1. Field of the Invention

The present invention relates to a composition, a spray, and a wiper.

### 2. Description of the Related Art

A virus is a structure that does not have a cell structure unlike microorganisms, such as bacteria and fungi having a cell structure, and has a genome enclosed in a shell protein called capsid. Depending on whether the genome is deoxyribonucleic acid (DNA) or ribonucleic acid (RNA), viruses are roughly classified into 2 types which are further classified into enveloped viruses that have the capsid surrounded by an envelope consisting of a lipid bilayer and non-enveloped viruses that have the capsid not being surrounded by an envelope. Specifically, the DNA-type enveloped viruses include human herpesvirus and hepatitis B virus, the DNA-type non-enveloped viruses include adenovirus and B19 virus, the RNA-type enveloped viruses include influenza virus and severe acute respiratory syndrome (SARS) coronavirus, and the RNA-type non-enveloped viruses include norovirus, poliovirus, and enterovirus.

In recent years, there has been a demand for a drug capable of inactivating viruses (particularly, norovirus) in a simpler way. For example, JP4975987B discloses a norovirus disinfectant solution which contains 0.05% to 0.5% by weight of a polyhexamethylene biguanide-based compound and 40% to 80% by weight of an alcohol and has a pH in a range of 9 to 12.

### SUMMARY OF THE INVENTION

The inventors of the present invention prepared the disinfectant solution composition described in JP4975987B, and examined the antiviral activity thereof on feline calicivirus (norovirus-related species that has genomic composition, capsid structure, and biochemical characteristics similar to those of norovirus and is thus currently most widely used as a virus alternative to norovirus). As a result, the inventors have revealed that the antiviral activity needs to be further improved.

An object of the present invention is to provide a composition having excellent antiviral activity.

Another object of the present invention is to provide a spray and a wiper that use the above composition.

In order to achieve the above objects, the inventors of the present invention conducted intensive studies. As a result, the inventors have found that the objects can be achieved by a composition having a specific formulation, and have accomplished the present invention.

That is, the inventors of the present invention have found that the above objects can be achieved by the following constitutions.

[1] An alkaline composition containing a compound having an amino group and at least one kind of functional group selected from the group consisting of an acidic group, a hydroxyl group, and a phenyl group and having a molecular weight equal to or higher than 85; and a solvent containing an alcohol.

[2] The composition described in [1], in which the compound is a compound represented by Formula (A) which will be described later or a salt thereof.

[3] The composition described in [1] or [2], in which the compound has 1 to 3 amino groups.

[4] The composition described in any one of [1] to [3], in which the functional group contains an acidic group.

[5] The composition described in any one of [1] to [4], in which the functional group contains a carboxylic acid group.

[6] The composition described in any one of [1] to [5], in which the compound has a structure in which an amino group and the functional group are bonded through 2 or more atoms.

[7] The composition described in any one of [1] to [6], in which the compound has a structure in which an amino group and the functional group are bonded through 3 or more atoms.

[8] The composition described in any one of [1] to [7], in which a content of the compound is 300 to 60,000 mg/L with respect to a total volume of the composition.

[9] The composition described in any one of [1] to [8], in which a content of the alcohol is higher than 40% by volume with respect to a total volume of the solvent.

[10] The composition described in any one of [1] to [9], which has a pH equal to or higher than 8.5 and equal to or lower than 14.0.

[11] The composition described in any one of [1] to [10], which is an antiviral composition.

[12] The composition described in any one of [1] to [11], which is an anti-norovirus composition.

[13] The composition described in any one of [1] to [12], which is a liquid agent.

[14] The composition described in any one of [1] to [12], which is a gel agent.

[15] A spray including a spray container and the composition described in any one of

[1] to [13] stored in the spray container.

[16] A wiper including a base fabric and the composition described in any one of [1] [14] with which the base fabric is impregnated.

[17] The wiper described in [16], in which a content of a cellulosic fiber is equal to or lower than 70% by mass with respect to a total mass of fibers constituting the base fabric.

[18] The wiper described in [16] or [17], in which a content of a cellulosic fiber is equal to or lower than 30% by mass with respect to a total mass of fibers constituting the base fabric.

[19] The wiper described in any one of [16] to [18], in which the base fabric substantially does not contain cellulosic fiber.

[20] The wiper described in any one of [16] to [19], in which the base fabric contains one or more kinds of synthetic fibers selected from the group consisting of a polyolefin fiber, a polyester fiber, a vinylon fiber, and a nylon fiber.

According to an aspect of the present invention, it is possible to provide a composition having excellent antiviral activity.

Furthermore, according to an aspect of the present invention, it is possible to provide a spray and a wiper that use the above composition.

### DESCRIPTION OF THE PREFERRED EMBODIMENTS

Hereinafter, the present invention will be specifically described.

The following constituents will be described based on the typical embodiments of the present invention in some cases, but the present invention is not limited to the embodiments.

In the present specification, the range of numerical values described using “to” means a range including the numerical values listed before and after “to” as the lower limit and the upper limit.

In the present specification, “(meth) acrylate” has a concept that includes either or both of acrylate and methacrylate.

In the present specification, in a case where there is a plurality of substituents and linking groups (hereinafter, described as substituents and the like) marked with specific reference signs, or in a case where a plurality of substituents and the like are simultaneously specified, the substituents and the like may be the same as or different from each other. The same is true of a case where the number of substituents and the like is specified.

Furthermore, in the present specification, in a case where there is no description regarding whether a group (atomic group) is substituted or unsubstituted, the group includes a group having no substituent and a group having a substituent. For example, “alkyl group” includes not only an alkyl group having no substituent (unsubstituted alkyl group) but also an alkyl group having a substituent (substituted alkyl group).

[Composition]

The composition according to an embodiment of the present invention is an alkaline composition containing a component A which will be described later and a solvent containing an alcohol.

The inventors of the present invention have found that the alkaline composition containing the component A, which will be described later, and a solvent containing an alcohol exhibits remarkably excellent antiviral activity even under the conditions closer to the actual environment such as the human intestinal environment (particularly, antiviral activity against feline calicivirus (norovirus-related species)).

In addition, it has been also confirmed that the composition according to an embodiment of the present invention also has excellent antimicrobial activity against microorganisms such as bacteria and fungi (for example, Escherichia coli, Staphylococcus, and the like).

Details of the mechanism thereof are unclear, but are assumed to be as below by the inventors of the present invention.

Some viruses and microorganisms, such as norovirus, have an infection pathway in which the viruses or microorganisms grow in the human intestine, spread to the outside as feces or vomitus, and infect others. Therefore, for an environmental disinfectant against these viruses and microorganisms, it is important that the ability to disinfect the viruses and microorganisms is not hindered by impurities such as proteins and a neutral buffer derived from a living body. The composition according to an embodiment of the present invention has an alkaline pH and uses a component A that exerts a buffering ability in an alkaline pH range. Presumably, for this reason, the impurities and neutral buffer described above may be inhibited from affecting the ability of the composition to disinfect the viruses and microorganisms.

Furthermore, the inventors of the present invention consider that the alcohol in the composition may also contribute to the inactivation of the virus. It is considered that due to the synergy of the above mechanisms, the composition according to an embodiment of the present invention may have remarkably excellent antiviral activity (particularly, antiviral activity against feline calicivirus (norovirus-related species)).

The composition according to an embodiment of the present invention has particularly excellent antiviral activity against feline calicivirus (norovirus-related species). Therefore, it is preferable that the composition be used as an anti-norovirus composition.

Hereinafter, each of the components contained in the composition according to an embodiment of the present invention will be specifically described.

[Component A]

The composition according to an embodiment of the present invention contains an amine compound which has an amino group and at least one kind of functional group selected from the group consisting of an acidic group, a hydroxyl group, and a phenyl group and has a molecular weight equal to or higher than 85 (hereinafter, the compound will be also described as “component A”).

The amino group contained in the component A may have a substituent. Therefore, the component A may be any of a primary amine, a secondary amine, and a tertiary amine.

The number of amino groups contained in a molecule of component A is not particularly limited, but is preferably 1 to 5, more preferably 1 to 3, and even more preferably 1 or 2.

The component A has at least one kind of functional group (hereinafter, also described as “functional group Q”) selected from the group consisting of an acidic group, a hydroxyl group, and a phenyl group. Examples of the acidic group include phosphorus atom-containing acid groups, such as a carboxylic acid group, a sulfonic acid group, and a phosphoric acid group, and salts of these. The component A may have one kind of functional group Q or two or more kinds of functional groups Q.

As the functional group Q, an acidic group is preferable, and a carboxylic acid group or a sulfonic acid group is more preferable. Among these, a carboxylic acid group is more preferable because it has better antiviral activity.

The number of functional groups Q contained in a molecule of the component A is not particularly limited, and may be 1 to 7. In a case where the functional group Q is an acidic group, the number of acidic groups contained in a molecule of the component A is preferably 1 to 5, more preferably 1 to 3, and even more preferably 1 or 2. In a case where the functional group Q is a hydroxyl group, the number of hydroxyl groups contained in a molecule of the component A is preferably 1 to 7, and more preferably 3 to 5. In a case where the functional group Q is a phenyl group, the number of phenyl groups contained in a molecule of the component A is preferably 1 or 2, and more preferably 1.

The molecular weight of the component A is equal to or higher than 85. In a case where the component A having a molecular weight equal to or higher than 85 is used, the antiviral activity of the composition can be improved. The upper limit of the molecular weight of the component A is not particularly limited, but is preferably equal to or lower than 300 and more preferably equal to or lower than 200.

As the component A, a compound represented by Formula (A) or a salt thereof is preferable.

{(Ra)₂N}ₘ-L-(Q)ₙ (A)

In Formula (A), Ra represents a hydrogen atom or a hydrocarbon group having 1 to 10 carbon atoms, Q represents one kind of functional group selected from the group consisting of an acidic group, a hydroxyl group, and a phenyl group, m represents an integer of 1 to 3, n represents an integer of 1 to 5, and L represents an (m+n)-valent organic group.

“Salt of the compound represented by Formula (A)” is a salt consisting of the compound represented by Formula (A) and the corresponding acid. The salt is represented by Formula (A1).

[{(Ra)₂N}ₘ-L-(Q)ₙ·HX] (A 1)

In Formula (A1), HX represents a protonic acid. An anion X⁻ constituting the protonic acid represented by HX is not particularly limited unless this anion inhibits the action of the component A. Examples of X⁻ include a halide ion, a sulfate anion, a hydrogen sulfate anion, a sulfonate anion, a carboxylate anion, a nitrate anion, a phosphate anion, a hydrogen phosphate anion, a dihydrogen phosphate anion, a phosphonate anion, and a borate anion. Among these, a halide ion, a sulfonate anion, or a carboxylate anion is preferable, a halide ion is more preferable, and a chloride ion is even more preferable.

In the present specification, in a case where “salt of the compound” is described for the compound represented by Formula (A), unless otherwise specified, “salt of the compound” means that the compound represented by Formula (A) includes a salt thereof, that is, the compound represented by Formula (A1).

Examples of the hydrocarbon group having 1 to 10 carbon atoms represented by Ra include a linear or branched alkyl group having 1 to 10 carbon atoms, a linear or branched alkenyl group having 2 to 10 carbon atoms, a linear or branched alkynyl group having 2 to 10 carbon atoms, a cycloalkyl group having 3 to 10 carbon atoms, and an aryl group having 6 to 10 carbon atoms.

Examples of the linear or branched alkyl group having 1 to 10 carbon atoms include a methyl group, an ethyl group, a n-propyl group, an isopropyl group, a n-butyl group, an isobutyl group, a sec-butyl group, a tert-butyl group, a n-pentyl group, an isopentyl group, a neopentyl group, a 1,1-dimethylpropyl group, a n-hexyl group, a 2-ethylhexyl group, an isohexyl group, a heptyl group, an octyl group, a 3,7-dimethyloctyl group, a nonyl group, and a decyl group.

Examples of the linear or branched alkenyl group having 2 to 10 carbon atoms include a vinyl group, a propenyl group, a butenyl group, a pentenyl group, a hexenyl group, a heptenyl group, an octenyl group, a nonenyl group, a decenyl group, a butadienyl group, a pentadienyl group, a hexadienyl group, and an octadienyl group.

Examples of the linear or branched alkynyl group having 2 to 10 carbon atoms include an ethynyl group, a propynyl group, a butynyl group, a pentynyl group, a hexynyl group, a heptynyl group, an octynyl group, a nonynyl group, and a decynyl group.

Examples of rings constituting the cycloalkyl group having 3 to 10 carbon atoms include cyclopropane, cyclobutane, cyclopentene, cyclopentadiene, cyclohexane, 2-isopropyl-5-methylcyclohexane, cyclohexene, cyclohexadiene, cycloheptane, cycloheptene, cycloheptadiene, cyclooctane, cyclooctene, cyclooctadiene, cyclooctatriene, cyclononane, cyclononene, cyclodecane, cyclodecene, cyclodecadiene, cyclodecatriene, and norbornane.

Examples of the aryl group having 6 to 10 carbon atoms include a phenyl group, a tolyl group, a xylyl group, and a naphthyl group.

Ra is preferably a hydrogen atom, an alkyl group having 1 to 4 carbon atoms, a cycloalkyl group having 4 to 6 carbon atoms, or a phenyl group, more preferably a hydrogen atom or an alkyl group having 1 to 3 carbon atoms, and even more preferably a hydrogen atom. It is preferable that at least one Ra in Formula (A) represent a hydrogen atom. It is more preferable that both of two Ra's in Formula (A) represent a hydrogen atom.

The hydrocarbon group having 1 to 10 carbon atoms represented by Ra may further have a substituent. Examples of the substituent include the groups listed in the substituent group W which will be described later. Furthermore, Ra may be bonded to L directly or through a linking group so as to form a ring structure.

In Formula (A), m is preferably 1 or 2, and more preferably 1.

(Substituent Group W)

Examples of the groups included in the substituent group W include a halogen atom (such as a fluorine atom, a chlorine atom, a bromine atom, or an iodine atom), a cyano group, a nitro group, an alkoxy group, an aryloxy group, a heterocyclic oxy group, a mercapto group, an alkylthio group, an arylthio group, a heterocyclic thio group, and a silyl group.

Furthermore, the groups listed in the substituent group W may be further substituted with the groups exemplified as groups belonging to the substituent group W. For example, an alkoxy group may be substituted with a halogen atom.

Q in Formula (A) has the same definition as the functional group Q described above, including preferred aspects thereof.

L is not particularly limited as long as it is an organic group having a valence equal to (m+n) which is the total number of {(Ra)₂N} and Q. Examples of L include a linear or branched aliphatic hydrocarbon group having 1 to 10 carbon atoms, an alicyclic hydrocarbon group having 3 to 10 carbon atoms, an aromatic hydrocarbon group having 6 to 10 carbon atoms, and a group obtained by combining these.

In a case where m+n equals 2, examples of the organic group (divalent organic group) include a linear or branched alkylene group having 1 to 10 carbon atoms, a linear or branched alkenylene group having 2 to 10 carbon atoms, a linear or branched alkynylene group having 2 to 10 carbon atoms, a cycloalkylene group having 3 to 10 carbon atoms, and an arylene group having 6 to 10 carbon atoms. Specific examples of these groups include groups obtained in a case where a certain hydrogen atom is removed from specific examples of the linear or branched alkyl group having 1 to 10 carbon atoms, the linear or branched alkenyl group having 2 to 10 carbon atoms, the linear or branched alkynyl group having 2 to 10 carbon atoms, the cycloalkyl group having 3 to 10 carbon atoms, and the aryl group having 6 to 10 carbon atoms represented by Ra described above.

The aforementioned divalent organic group is preferably a linear or branched alkylene group having 1 to 10 carbon atoms, more preferably a linear or branched alkylene group having 1 to 6 carbon atoms, and even more preferably a linear alkylene group having 2 to 5 carbon atoms.

In a case where (m+n) is equal to or greater than 3, examples of the corresponding organic group having a valence equal to or higher than 3 include groups obtained in a case where certain (m+n−2) hydrogen atoms are removed from the aforementioned divalent organic group. The same shall be applied to the suitable aspects thereof.

The (m+n)-valent organic group represented by L may further have a substituent in addition to {(Ra)₂N} and Q in Formula (A). Examples of the substituent include the groups listed in the substituent group W described above.

Furthermore, the organic group may have a heteroatom. The type of the heteroatom is not particularly limited, and examples thereof include an oxygen atom, a nitrogen atom, and a sulfur atom. Examples of the organic group having a heteroatom include a group having a structure established in a case where a single bond in the aliphatic hydrocarbon group is substituted with a linking group selected from the group consisting of —O—, —CO—, —COO—, —S—, and —NRb-. Rb represents a hydrogen atom or an alkyl group having 1 to 6 carbon atoms. Rb is preferably a hydrogen atom or an alkyl group having 1 to 3 carbon atoms, and more preferably a hydrogen atom.

The component A preferably has a structure in which an amino group and the functional group Q are bonded through 2 or more atoms, more preferably has a structure in which an amino group and the functional group Q are bonded through 3 or more atoms, and even more preferably has a structure in which an amino group and the functional group Q are bonded through 4 or more atoms, because then the antiviral activity is further improved, the corrosion of metals can be inhibited, and the composition can be applied to a wider variety of objects. Examples of the metals include aluminum, copper, and brass.

In a case where the component A contains a plurality of amino groups and/or a plurality of functional groups Q, and the number of atoms constituting a bond between each of the amino groups and each of the functional groups Q is calculated based on the combination of the amino group and the functional group Q, the component A preferably has a structure in which the maximum number of atoms calculated as above is equal to or greater than 2, more preferably has a structure in which the maximum number of atoms calculated as above is equal to or greater than 3, and even more preferably has a structure in which the maximum number of atoms calculated as above is equal to or greater than 4.

In a case where the number of atoms intervening between an amino group and the functional group Q in the component A is equal to or greater than 2 (more preferably equal to or greater than 3), the corrosion inhibition effect on metals is improved through a certain mechanism. Although the mechanism is not limited to any theory, presumably, the greater the number of atoms is, the lower the stability of the complex formed of the component A and metal cations may be, and the corrosion inhibition effect may be improved.

As the component (A), for example, a compound represented by Formula (B1) is preferable.

{(Ra)₂N }-(CH₂)ⱼ-Q (B1)

In Formula (B1), Ra and Q have the same definitions as Ra and Q in Formula (A), and j represents an integer of 2 to 8.

Ra in Formula (B1) is preferably a hydrogen atom, an alkyl group having 1 to 3 carbon atoms, or a cycloalkyl group having 4 to 6 carbon atoms, more preferably a hydrogen atom, a methyl group, an ethyl group, or a cyclohexyl group, and even more preferably a hydrogen atom.

Q in Formula (B1) is preferably a carboxylic acid group, a sulfonic acid group, or a phenyl group, more preferably a carboxylic acid group or a sulfonic acid group, and even more preferably a carboxylic acid group.

j is preferably an integer of 2 to 5. In view of further improving the antiviral activity and further improving the corrosion inhibition effect on metals, j is more preferably an integer of 3 to 5.

As the component (A), for example, a compound represented by Formula (B2) is also preferable.

{(Ra)₂N}-CH₂-(CH(X))ₖ-Q (B2)

In Formula (B2), X represents a hydrogen atom or Q, and at least one X represents Q. Ra and Q in Formula (B2) have the same definitions as Ra and Q in Formula (A). k represents an integer of 1 to 5.

Ra in Formula (B2) is preferably a hydrogen atom or an alkyl group having 1 to 3 carbon atoms, and more preferably a hydrogen atom or a methyl group.

Q in Formula (B2) is preferably an acidic group or a hydroxyl group, and more preferably a hydroxyl group.

k is preferably an integer of 2 to 5, and more preferably an integer of 3 to 5.

The number of Q's contained in the compound represented by Formula (B2) is preferably 2 to 5, and more preferably 3 to 5.

As the component (A), for example, a compound represented by Formula (B3) is also preferable.

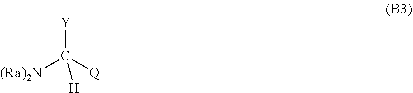

In Formula (B3), Ra and Q have the same definitions as Ra and Q in Formula (A), and Y represents an alkyl group or alkenyl group having 1 to 5 carbon atoms that may have a substituent.

Q in Formula (B3) is preferably a carboxylic acid group, a sulfonic acid group, or a phenyl group, and more preferably a carboxylic acid group.

Y is preferably an alkyl group having 2 to 5 carbon atoms that may have a substituent, and more preferably an alkyl group having 3 to 5 carbon atoms that may have a substituent.

Examples of the substituent that the alkyl group or alkenyl group represented by Y has include the groups included in the aforementioned substituent group W, {(Ra)₂N}, and Q. Y is preferably an alkyl group having 2 to 5 carbon atoms having no substituent or an alkyl group having 2 to 5 carbon atoms having at least one kind of substituent selected from the group consisting of an amino group, a phenyl group, and an alkylthio group. In view of further improving the antiviral activity, Y is more preferably an alkyl group having 3 to 5 carbon atoms having an amino group.

As the component A, for example, a compound represented by Formula (A) is also preferable in which Ra represents a hydrogen atom, an alkyl group having 1 to 3 carbon atoms, or a cyclohexyl group, Q represents an acidic group (more preferably a carboxylic acid group), m represents 1 or 2 (more preferably 1), n represents 1, L represents a linear alkylene group having 2 to 5 carbon atoms, and one end of L representing the linear alkylene group is bonded to at least one {(Ra)₂N} and the other end thereof is bonded to Q.

Furthermore, the component A is preferably a compound having an acid dissociation constant (pKa) of 9.0 to 12.5, and more a compound having a pKa of 9.5 to 12.0, because these compounds exhibit higher antiviral activity in the suitable pH range of the composition that will be described later.

The method of measuring the pKa of the component A is not particularly limited. For example, the pKa may be measured by acid-base titration. In a case where the component A is a commercially available product, the value of pKa described in the catalog of the commercially available product may be adopted.

Specific examples of the component A will be shown below. However, the component A is not limited to the following specific examples. The abbreviations in the examples mean the following compounds.

“CAPS”: N-cyclohexyl-3-aminopropanesulfonic acid

“CAPS O”: 3-(cyclohexylamino)-2-hydroxy-1-propanesulfonic acid

“CHES”: 2-(cyclohexylamino)ethanesulfonic acid

“AMPSO”: N-(1,1-dimethyl-2-hydroxyethyl)-3-amino-2-hydroxypropanesulfonic acid

“CABS”: 4-(cyclohexylamino)-1-butanesulfonic acid

“MOBS”: 4-(N-morpholino)butanesulfonic acid

“DIPSO”: 3-(N,N-bis[2-hydroxyethyl]amino)-2-hydroxypropanesulfonic acid

“POPSO”: piperazine-1,4-bis(2-hydroxypropanesulfonic acid)

“EPPS”: 4-(2-hydroxyethyl)-1-piperazinepropanesulfonic acid

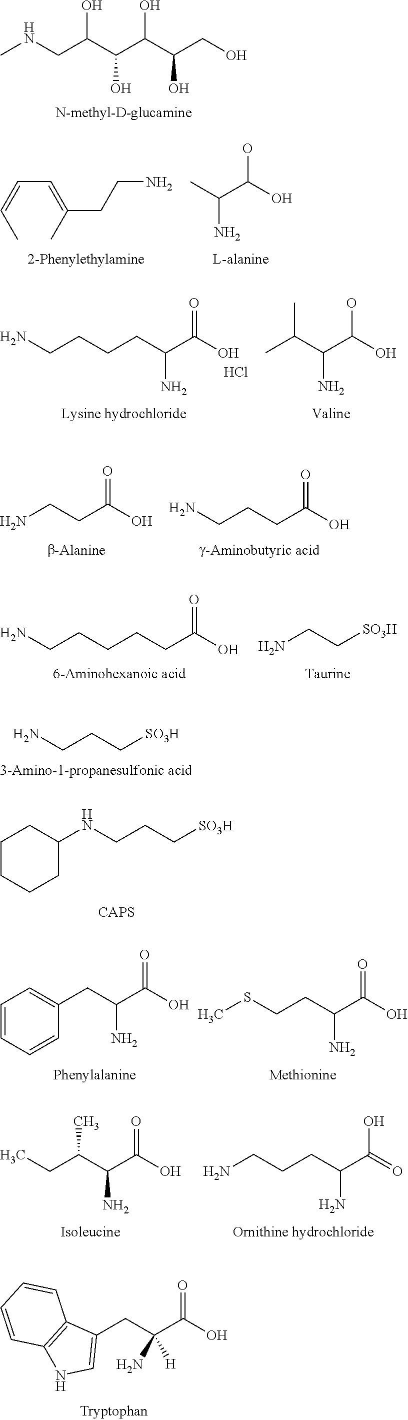

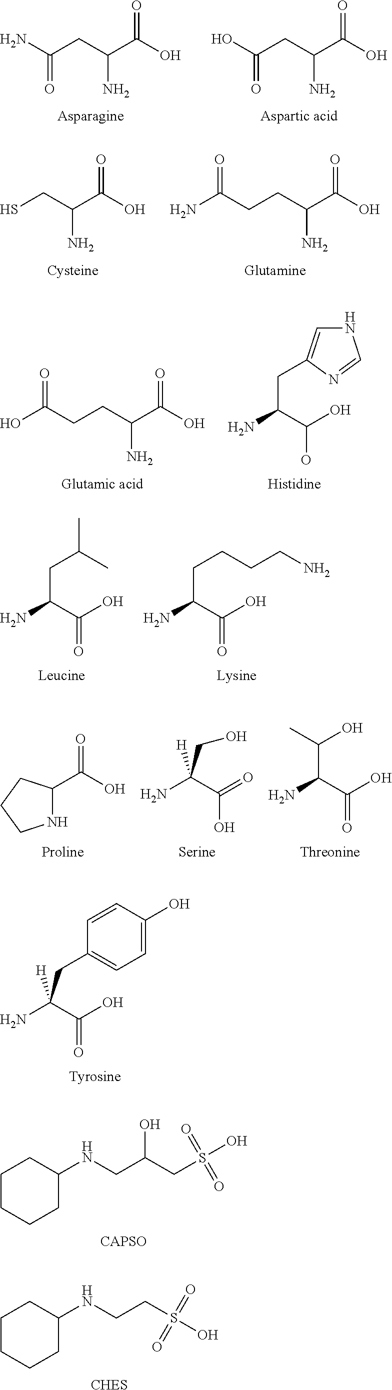

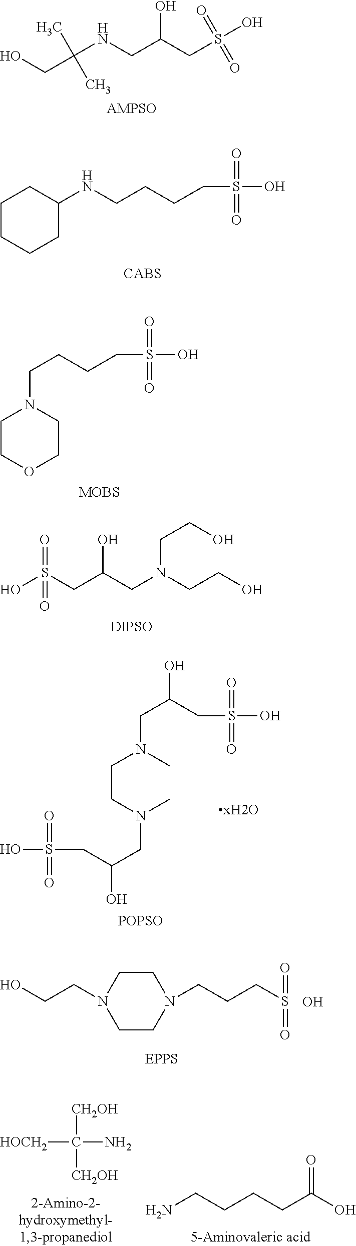

In view of further improving the antiviral activity, the content (mass) of the component A (total content in a case where the composition contains two or more kinds of components A) with respect to the total volume of the composition is preferably equal to or higher than 300 mg/L, more preferably equal to or higher than 1,000 mg/L, and even more preferably equal to or higher than 3,000 mg/L. In view of further improving wiping properties of the composition, the upper limit of the content of the component A with respect to the total volume of the composition is preferably equal to or lower than 60,000 mg/L, more preferably equal to or lower than 30,000 mg/L, even more preferably equal to or lower than 15,000 mg/L, and particularly preferably equal to or lower than 6,000 mg/L.

The wiping properties of the composition means the extent to which the component derived from the composition remains on an object after the composition applied to the object is wiped with a substrate such as a wiper. In a case where the wiping properties of the composition are poor, wiping marks of the component derived from the composition remain on the object, which leads to a concern that the object may look unattractive and need to be wiped again.

[Solvent]

The composition according to an embodiment of the present invention contains a solvent.

The content of the solvent in the composition is not particularly limited. The content of the solvent (total content in a case where the composition contains two or more kinds of solvents) with respect to the total mass of the composition is preferably 0.5% to 99.9% by mass, more preferably 10% to 99.8% by mass, even more preferably 50% to 99.8% by mass, and particularly preferably 80% to 99.8% by mass.

<Alcohol>

The composition according to an embodiment of the present invention contains alcohol as a solvent.

In the present specification, an alcohol means a compound having an alcoholic hydroxyl group, and does not include a compound having a phenolic hydroxyl group.

The alcohol is not particularly limited, but is preferably a linear, branched, or cyclic alcohol (including an ether alcohol) having 1 to 20 carbon atoms. Examples of the linear, branched, or cyclic alcohol having 1 to 20 carbon atoms include methanol, ethanol, n-propanol, isopropanol, polyethylene glycol, propylene glycol acetate monoester, n-butanol, 2-butanol, i-butanol, t-butanol, butane-1,3-diol, diethylene glycol, diethylene glycol monoethyl ether, diethylene glycol monopropyl ether, dipropylene glycol, n-pentanol, 2-pentanol, 3-pentanol, t-amyl alcohol, isoamyl alcohol, 2-methylbutanol, 3-methyl-2-butanol, 3-methyl-2-butenol, 3-methyl-3-butanol, 1-penten-3-ol, n-hexanol, capryl alcohol, 2-ethyl-1-hexanol, decanol, linalol, geraniol, lauryl alcohol, myristyl alcohol, benzyl alcohol, phenylethyl alcohol, cinnamyl alcohol, 3-methoxypropanol, methoxymethoxyethanol, ethylene glycol, ethylene glycol mono-n-butyl ether, diethylene glycol mono-n-butyl ether, triethylene glycol mono-n-butyl ether, tetraethylene glycol mono-n-butyl ether, dipropylene glycol monobutyl ether, citronellol, terpineol, hydroxycitronellal, hydroxycitronellal dimethyl acetal, ethylene glycol monomethyl ether, ethylene glycol monoethyl ether, propylene glycol monomethyl ether, propylene glycol monoethyl ether, diacetone alcohol, ethylene glycol monoisopropyl ether, and diethylene glycol monomethyl ether.

As the aforementioned alcohol, from the viewpoint of safety, food additives are preferable. Among these, methanol, ethanol, propanol, isopropanol, polyethylene glycol, propylene glycol acetate monoester, n-butanol, 2-butanol, butane-1,3-diol, diethylene glycol, diethylene glycol monoethyl ether, diethylene glycol monopropyl ether, dipropylene glycol, 2-methyl-1-butanol, 1-decanol, 1-p enten-3-ol, 2-ethyl-1-hexanol, 2-p entanol, 3-p entanol, 3-methyl-2-butanol, 3-methyl-2-butenol, 3-methyl-3-butanol, iso amyl alcohol, isobutanol, benzyl alcohol, citronellol, terpineol, hydroxycitronellal, or hydroxycitronellal dimethyl acetal is more preferable.

It is preferable that the composition according to an embodiment of the present invention contain, as alcohols, a first alcohol having 2 or less carbon atoms and a second alcohol having 3 or more carbon atoms, because then the variation in the value of antiviral activity is further reduced.

The second alcohol is considered to be more lipophilic than the first alcohol and make it easy to physically remove viruses and virus-carrying contaminants. Therefore, it is considered that in a case where a wiper is impregnated with the composition containing the first alcohol and the second alcohol and used for wiping, the variation in the value of antiviral activity may be further reduced. The second alcohol is assumed to have a higher surface activation function compared to the first alcohol. It is considered that for this reason, the synergy with the component A may be further enhanced, and the antiviral activity may be further improved.

The mixing ratio of the first alcohol and the second alcohol is not particularly limited. In view of further reducing the variation in the antiviral activity, the volume ratio of the content of the second alcohol to the content of the first alcohol (volume of second alcohol/volume of first alcohol) is preferably equal to or higher than 0.001, and more preferably equal to or higher than 0.01. The upper limit of the ratio is not particularly limited, but is equal to or lower than 5, for example. The upper limit is preferably equal to or lower than 1.5.

Hereinafter, the first alcohol and the second alcohol will be specifically described.

(First Alcohol)

The first alcohol is an alcohol having 2 or less carbon atoms. More specifically, examples thereof include methanol and ethanol. Among these, ethanol is preferable. As the first alcohol, methanol or ethanol may be used singly, or both the methanol and ethanol may be used.

The content of the first alcohol (total content in a case where both the methanol and ethanol are used) is not particularly limited, but is preferably 40% to 99% by volume with respect to the total volume of the solvent. Particularly, in view of further improving the antiviral activity, the content of the first alcohol is more preferably 50% to 95% by volume, and even more preferably 60% to 90% by volume.

(Second Alcohol)

The second alcohol is an alcohol having 3 or more carbon atoms. The second alcohol is not particularly limited as long as it has 3 or more carbon atoms. Examples thereof include a linear, branched, or cyclic alcohol (including an ether alcohol).

The number of carbon atoms in the second alcohol is preferably 3 to 10, more preferably 3 to 7, and even more preferably 3 to 5. The second alcohol is preferably linear or branched, and more preferably linear. Furthermore, as the second alcohol, a monohydric alcohol or a dihydric alcohol is preferable, and a monohydric alcohol is more preferable.

One kind of second alcohol may be used singly, or two or more kinds of second alcohols may be used.

The content of the second alcohol (total content in a case where two or more kinds of second alcohols are used) is not particularly limited. The content of the second alcohol with respect to the total volume of the solvent is preferably equal to or higher than 0.1% by volume, and more preferably equal to or higher than 1% by volume. The upper limit of the content of the second alcohol is not particularly limited. The upper limit with respect to the total volume of the solvent is preferably equal to or lower than 20% by volume, and more preferably equal to or lower than 5% by volume.

In view of further improving the antiviral activity, the content of the alcohol contained in the composition (total content in a case where the composition contains two or more kinds of alcohols) with respect to the total volume of the solvent is more preferably higher than 40% by volume, more preferably equal to or higher than 50% by volume, even more preferably higher than 70% by volume, and particularly preferably higher than 80% by volume. In a case where the content of the alcohol is within the above range, the antiviral activity of the composition can be further improved.

Furthermore, the content of the alcohol with respect to the total volume of the solvent is preferably lower than 100% by volume, and more preferably equal to or lower than 99% by volume. The content of the alcohol may be 100% by volume with respect to the total volume of the solvent. In other words, the solvent may only contain an alcohol.

In view of further improving the antiviral activity, the content of the alcohol with respect to the total mass of the composition is preferably higher than 50% by mass and lower than 100% by mass, and more preferably 80% to 99% by mass.

<Solvent Other than Alcohol>

The composition according to an embodiment of the present invention may contain a solvent other than an alcohol. Examples of the solvent other than an alcohol include water and an organic solvent (excluding an alcohol).

It is preferable that the present composition contain water. Particularly, in view of storage stability of the composition, the water is more preferably deionized water, distilled water, filtered water, or pure water.

In a case where the solvent contains water, the content of water is not particularly limited, and may be the rest of the solvent other than the aforementioned alcohol. The content of water with respect to the total volume of the solvent is preferably equal to or higher than 0.1% by volume, more preferably equal to or higher than 1% by volume, and even more preferably equal to or higher than 5% by volume. The upper limit of the content of water is not particularly limited, and is preferably equal to or lower than 20% by volume.

The organic solvent is not particularly limited, and examples thereof include acetone, methyl ethyl ketone, cyclohexane, benzene, ethyl acetate, isoamyl acetate, isopropyl acetate, geranyl acetate, cyclohexyl acetate, citronellyl acetate, cinnamyl acetate, terpinyl acetate, phenylethyl acetate, butyl acetate, benzyl acetate, menthyl acetate, linalyl acetate, butyric acid, ethyl butyrate, butyl butyrate, isoamyl butyrate, cyclohexyl butyrate, ethylene dichloride, tetrahydrofuran, toluene, ethylene glycol dimethyl ether, acetylacetone, cyclohexanone, ethylene glycol monomethyl ether acetate, ethylene glycol ethyl ether acetate, ethylene glycol monobutyl ether acetate, diethylene glycol dimethyl ether, diethylene glycol diethyl ether, propylene glycol monomethyl ether acetate, 2-methylpropanal, 2-methylbutyraldehyde, 3-methyl-2-butenal, 3-methylbutanal, L-perillaldehyde, acetaldehyde, ethyl acetoacetate, isovaleraldehyde, isobutanal, isopropyl myristerate, isoamyl isovalerate, ethyl isovalerate, ethyl lactate, ethyl heptanoate, octanal, ethyl octanoate, octanoic acid, octyl aldehyde, formic acid, isoamyl formate, geranyl formate, citronellyl formate, silicate aldehyde, ethyl silicate, methyl silicate, citral, citronellal, diisopropyl ether, diisopropyl disulfide, diisopropyl disulfide, diethyl ether, diethyl tartrate, diethyl pyrocarbonate, decanal, ethyl decanoate, triacetin, triethyl citrate, toluene, nonalactone, valeraldehyde, paramethylacetophenone, paramethoxybenzaldehyde, castor oil, isoamyl phenyl acetate, isobutyl phenyl acetate, ethyl phenyl acetate, butanal, propionaldehyde, propionic acid, isoamyl propionate, ethyl propionate, benzyl propionate, hexane, heptane, benzaldehyde, eucalyptol, ionone, a-amylcinnamaldehyde, brominated vegetable oil, acetic acid, dimethyl dicarbonate, thermally oxidized soybean oil, esters of thermally oxidized soybean oil and glycerin, and liquid paraffin.

Among these, from the viewpoint of safety, food additives are preferable, and acetone, methyl ethyl ketone, ethyl acetate, isoamyl acetate, isopropyl acetate, geranyl acetate, cyclohexyl acetate, citronellyl acetate, cinnamyl acetate, terpinyl acetate, phenylethyl acetate, butyl acetate, benzyl acetate, menthyl acetate, linalyl acetate, butyric acid, ethyl butyrate, butyl butyrate, isoamyl butyrate, cyclohexyl butyrate, 2-methylpropanal, 2-methylbutyraldehyde, 3-methyl-2-butenal, 3-methylbutanal, 1-perillaldehyde, acetaldehyde, ethyl acetoacetate, isovaleraldehyde, isobutanal, isopropyl myristerate, isoamyl isovalerate, ethyl isovalerate, ethyl lactate, ethyl heptanoate, octanal, octanoic acid, ethyl octanoate, octyl aldehyde, formic acid, isoamyl formate, geranyl formate, citronellyl formate, silicate aldehyde, ethyl silicate, methyl silicate, citral, citronellal, diisopropyl ether, diisopropyl disulfide, diisopropyl disulfide, diethyl ether, diethyl tartrate, diethyl pyrocarbonate, decanal, ethyl decanoate, triacetin, triethyl citrate, toluene, nonalactone, valeraldehyde, paramethylacetophenone, paramethoxybenzaldehyde, castor oil, isoamyl phenyl acetate, isobutyl phenyl acetate, ethyl phenyl acetate, butanal, propionaldehyde, propionic acid, isoamyl propionate, ethyl propionate, benzyl propionate, hexane, heptane, benzaldehyde, eucalyptol, ionone, a-amylcinnamaldehyde, brominated vegetable oil, acetic acid, dimethyl dicarbonate, thermally oxidized soybean oil, esters of thermally oxidized soybean oil and glycerin, or liquid paraffin is more preferable.

[PH of Composition]

The composition according to an embodiment of the present invention is alkaline. In the present specification, “alkaline” means that the pH of an aqueous composition is equal to or higher than 8.0.

In view of further improving the antiviral activity, the pH of the composition is preferably equal to or higher than 8.5, more preferably higher than 9.5, even more preferably equal to or higher than 10.0, and particularly preferably higher than 10.5.

Furthermore, the pH of the composition is preferably equal to or lower than 14.0 because then the safety is further improved, and more preferably equal to or lower than 12.0 because then the corrosion of metals can be inhibited and the composition can be applied to a wider variety of objects. Examples of the metals include aluminum, copper, and brass.

The method of adjusting the pH of the composition is not particularly limited. For example, the pH adjuster which will be described later may be added to the composition so that the pH is adjusted to the above range.

The method of measuring the pH is not particularly limited. For example, the pH can be measured using a desktop pH meter “F-72S” (manufactured by HORIBA, Ltd.) using a pH electrode “6337-10D” (manufactured by HORIBA, Ltd.). The measurement method will be specifically described later.

In the present specification, pH means a value at 25° C.

[Optional Component]

As long as the effects of the present invention are brought about, the composition according to an embodiment of the present invention may contain components other than the above. The optional components are not particularly limited. Examples thereof include a pH adjuster, a bactericide, a disinfectant, a fungicide, a surfactant, an antioxidant, an ultraviolet absorber, a chelating agent, a moisturizer, thickener, gelling agent, a preservative, a fragrance, and a coloring agent. Particularly, in view of further improving the antiviral activity, the composition according to an embodiment of the present invention preferably contains a bactericide, a disinfectant, a fungicide, a surfactant, or an antioxidant, and more preferably contains a quaternary ammonium salt (for example, benzalkonium chloride or the like), a surfactant, or an antioxidant.

<pH Adjuster>

The composition according to an embodiment of the present invention may contain a pH adjuster.

The pH adjuster is not particularly limited. Examples thereof include a metal alkoxide (for example, sodium methoxide, sodium ethoxide, or the like), a metal oxide (for example, calcium oxide, magnesium oxide, or the like), a hydrogen carbonate (such as ammonium hydrogen carbonate, sodium hydrogen carbonate, potassium hydrogen carbonate, or calcium hydrogen carbonate), a metal hydroxide (such as calcium hydroxide, magnesium hydroxide, potassium hydroxide, sodium hydroxide, lithium hydroxide, aluminum hydroxide, rubidium hydroxide, cesium hydroxide, strontium hydroxide, barium hydroxide, europium (II) hydroxide, or thallium (I) hydroxide), a carbonate (such as ammonium carbonate, potassium carbonate, calcium carbonate, sodium carbonate, magnesium carbonate, or cesium carbonate), a quaternary ammonium hydroxide, an organic base (a guanidine derivative, diazabicycloundecene, or diazabicyclononene), a phosphazene base, and a proazaphosphatrane base.

As the pH adjuster, from the viewpoint of safety, pH adjusters used as food additives are preferable, and sodium methoxide, calcium oxide, magnesium oxide, ammonium hydrogen carbonate, sodium hydrogen carbonate, potassium hydrogen carbonate, calcium hydrogen carbonate, calcium hydroxide, magnesium hydroxide, potassium hydroxide, sodium hydroxide, ammonium carbonate, potassium carbonate, calcium carbonate, sodium carbonate, or magnesium carbonate is more preferable.

One kind of pH adjuster may be used singly, or two or more kinds of pH adjusters may be used in combination.

There is no fixed limit on the content of the pH adjuster (total content in a case where the composition contains two or more kinds of pH adjusters) because the content of the pH adjuster varies with the type and content of the component A. For example, the content of the pH adjuster with respect to the total volume of the composition is preferably 10 to 300,000 mg/L, more preferably 50 to 200,000 mg/L, and even more preferably 100 to 100,000 mg/L.

<Bactericide, Disinfectant, and Fungicide>

The composition according to an embodiment of the present invention may further contain a drug selected from the group consisting of a bactericide, a disinfectant, and a fungicide.

The bactericide, the disinfectant, and the fungicide are not particularly limited. Examples thereof include a quaternary ammonium salt, a metal-containing antibacterial agent, a photocatalyst, an aldehyde-based compound, an iodine-based compound, a biguanide compound, and an acrinol hydrate (for example, 6,9-diamino-2-ethoxyacridine lactate monohydrate).

(Quaternary Ammonium Salt)

The quaternary ammonium salt is not particularly limited, and examples thereof include compounds represented by Formulas (2) to (5).

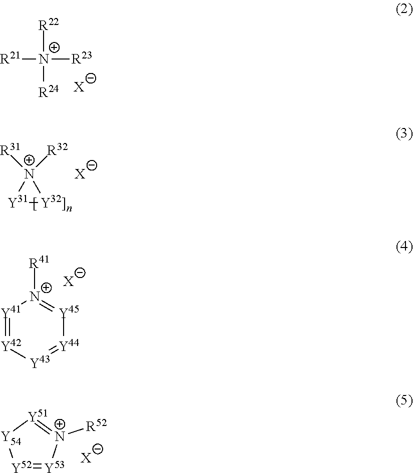

In Formula (2), R²¹ to R²⁴ each independently represent an aliphatic hydrocarbon group, an aryl group, an aralkyl group, or a heteroaryl group.

The aliphatic hydrocarbon group represented by R²¹ to R²⁴ may be linear, branched, or cyclic.

In the aliphatic hydrocarbon group represented by R²¹ to R²⁴, —CH₂— may be substituted with a heteroatom. The type of the heteroatom is not particularly limited, and examples thereof include an oxygen atom, a nitrogen atom, a sulfur atom, a selenium atom, and a tellurium atom. Particularly, in view of further improving the antiviral activity, it is preferable that the heteroatom be incorporated into the compound in the form of —Y¹—, —N(Ra)—, —C(═Y²)—, —CON(Rb)—, —C(═Y³)Y⁴—, —SOt—, —SO₂N(Rc)—, or a group obtained by combining these.

Y¹ to Y⁴ are each independently selected from the group consisting of an oxygen atom, a sulfur atom, a selenium atom, and a tellurium atom. Among these, in view of greater ease of handling, an oxygen atom or a sulfur atom is preferable. t represents an integer of 1 to 3. Ra, Rb, and Rc in the above formulas each independently represent a hydrogen atom or an alkyl group having 1 to 10 carbon atoms.

Examples of the aliphatic hydrocarbon group represented by R²¹ to R²⁴ include an alkyl group (preferably having 1 to 30 carbon atoms and more preferably having 1 to 20 carbon atoms), an alkenyl group (preferably having 2 to 30 carbon atoms and more preferably having 2 to 20 carbon atoms), and an alkynyl group (preferably having 2 to 30 carbon atoms and more preferably having 2 to 20 carbon atoms). Among these, an alkyl group is preferable.

Examples of the aryl group represented by R²¹ to R²⁴ include an aryl group having 6 to 10 carbon atoms. Examples of the aryl group include a phenyl group, a tolyl group, a xylyl group, and a naphthyl group.

The aralkyl group represented by R²′ to R²⁴ is not particularly limited, and is preferably an aralkyl group having 7 to 15 carbon atoms. Examples of the aralkyl group having 7 to 15 carbon atoms include a benzyl group, a phenethyl group, a 1-naphthylmethyl group, a 1-(1-naphthyl)ethyl group, a triphenylmethyl group, and a pyrenylmethyl group.

As the heteroaryl group represented by R²¹ to R²⁴, a heteroaryl group having 3 to 12 carbon atoms is preferable. Examples of the heteroaryl group having 3 to 12 carbon atoms include a furyl group, a thiofryl group, a pyridyl group, a pyrazole group, an imidazolyl group, a benzimidazolyl group, an indolyl group, a quinolyl group, an isoquinolyl group, a purine group, a pyrimidyl group, a pyrazyl group, an oxazolyl group, a thiazolyl group, a triazyl group, a carbazolyl group, a quinoxalyl group, and a thiazine group.

The aliphatic hydrocarbon group, aryl group, aralkyl group, and heteroaryl group represented by R²¹ to R²⁴ may further have a substituent. Examples of the substituent include the groups listed in the substituent Group W described above.

X⁻ represents a monovalent anion other than a hydroxide ion.

Examples of X⁻ include a halide ion (for example, F⁻, Cl⁻, Br⁻, I⁻, Br₃⁻, Br₂Cl⁻, I₃⁻, IBr₂⁻, Cl₂Br⁻, HF₂⁻, H₂F₃⁻, AuBr₂⁻, AuCl₂⁻, AuI₂⁻, and FeCl₄⁻), a carboxylate anion, a cyanide anion, a sulfonimide anion (N⁻(SO₂R)₂: R is a fluorine atom, a hydrocarbon group (for example, an alkyl group having 1 to 20 carbon atoms), or a perfluorohydrocarbon group (for example, a perfluoroalkyl group having 1 to 20 carbon atoms)), a borohydride anion, a dichloroiodate anion, a tetrafluoroborate anion, a hexafluorophosphate anion, a perchlorate anion, a sulfate anion, a hydrogen sulfate anion, a nitrate anion, a dicyanamide anion [N⁻(CN)₂], an azide anion (N₃⁻), an alkane or aryl sulfonate anion, a perfluoroalkane or aryl sulfonate anion, an alkyl or aryl sulfate anion (ROSO₃⁻: R represents an alkyl group having 1 to 20 carbon atoms or an aryl group having 6 to 18 carbon atoms), an alkyl or aryl phosphate anion ((RO)₂PO₂⁻: R each independently represents an alkyl group having 1 to 20 carbon atoms or an aryl group having 6 to 18 carbon atoms), a thiocyanide anion (S⁻CN), a triacetoxyborohydride anion, a perruthenate anion (RuO₄⁻), Cu(CF₃)₄⁻, C(CN)₃⁻, and CF₃BF₃⁻.

Examples of the compound represented by Formula (2) will be shown below, but the present invention is not limited thereto.

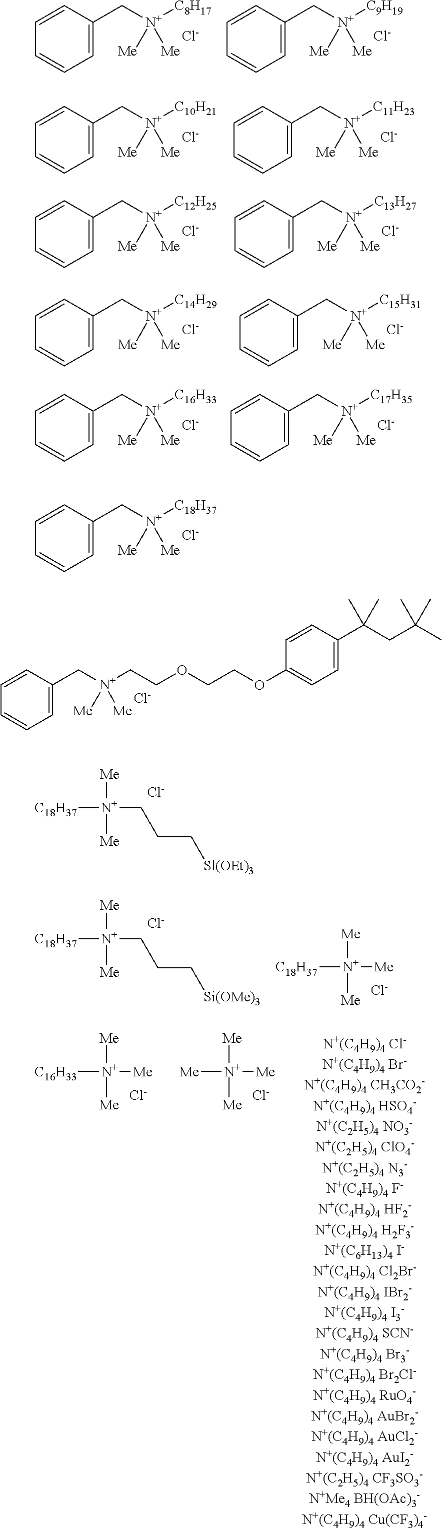

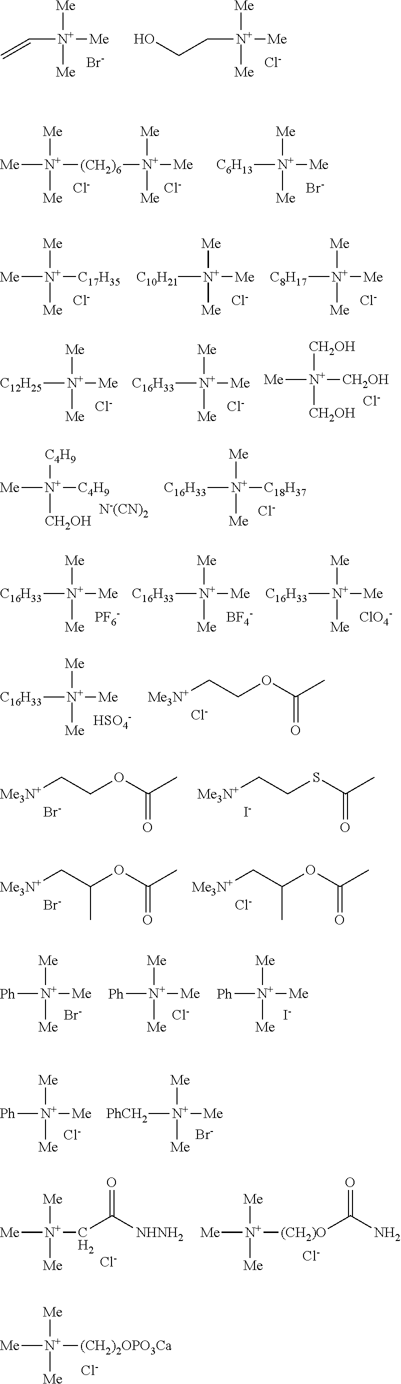

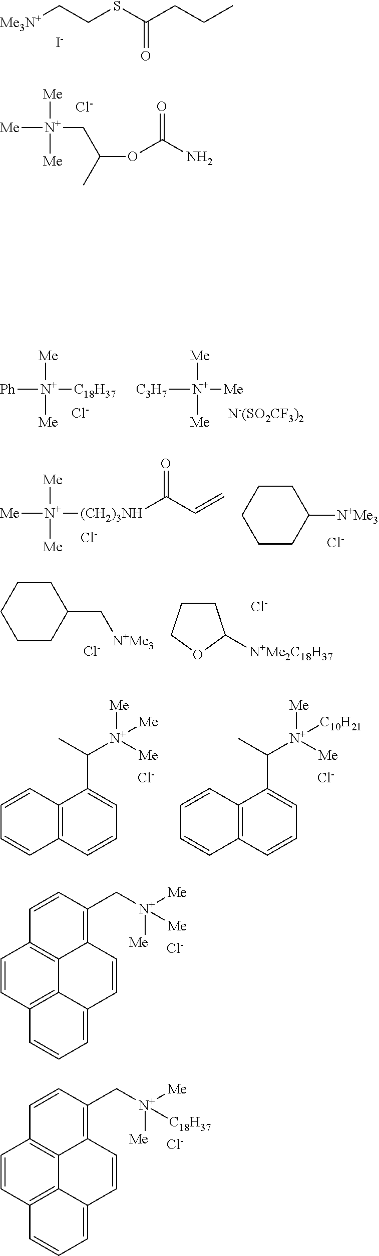

In Formula (3), X⁻ has the same definition as X⁻ in Formula (2). The suitable aspects of X⁻ in Formula (3) are the same as the suitable aspects of X⁻ in Formula (2).

R³¹ and R³² have the same definitions as R²¹ to R²⁴ in Formula (2). The suitable aspects of R³¹ and R³² are the same as the suitable aspects of R²¹ to R²⁴.

Y³¹ and Y³² each independently represent —C(R³³)₂—, —NR³⁴—, —O—, —CO—, —CO₂—, —S—, —SO—, or —SO₂—. In a case where there is a plurality of Y³²'s in Formula (2), the plurality of Y³²'s may be the same as or different from each other.

R³³ represents a hydrogen atom or a monovalent organic group selected from the group consisting of an aliphatic hydrocarbon group, an aryl group, an aralkyl group, a heteroaryl group, and a halogen atom.

R³⁴ represents a hydrogen atom or a monovalent organic group selected from the group consisting of an aliphatic hydrocarbon group, an aryl group, an aralkyl group, and a heteroaryl group.

The aliphatic hydrocarbon group, aryl group, aralkyl group, and heteroaryl group represented by R³³ and R³⁴ have the same definitions as the aliphatic hydrocarbon group, aryl group, aralkyl group, and heteroaryl group represented by R²¹ to R²⁴ in Formula (2). The suitable aspects of the groups represented by R³³ and R³⁴ are the same as the suitable aspects of the groups represented by R²¹ to R²⁴.

Examples of the halogen atom represented by R³³ and R³⁴ include a fluorine atom, a chlorine atom, a bromine atom, and an iodine atom.

The aliphatic hydrocarbon group, aryl group, aralkyl group, or heteroaryl group represented by R³³ and R³⁴ may further have a substituent. Examples of the substituent include those listed in the substituent group W described above.

In a case where Y³¹ or Y³² represents —C(R³³)₂— or —NR³⁴—, the monovalent organic group represented by R³¹ may be linked to R³³ or R³⁴ to form an aromatic or non-aromatic ring.

Furthermore, R³¹ and R³² may be linked to each other to form an aromatic or non-aromatic ring.

n represents an integer of 1 to 18.

Examples of the compound represented by Formula (3) will be shown below, but the present invention is not limited thereto.

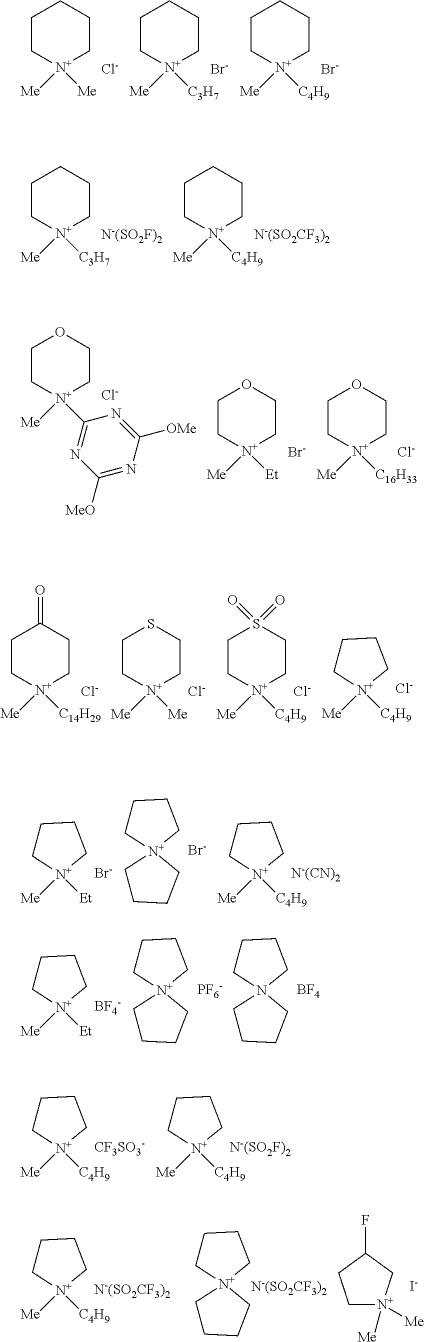

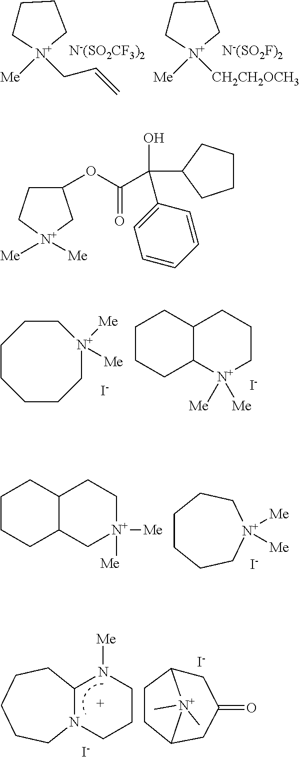

In Formula (4), X⁻ has the same definition as X⁻ in Formula (2). The suitable aspects of X⁻ in Formula (4) are the same as the suitable aspects of X⁻ in Formula (2).

R⁴¹ has the same definition as R²¹ to R²⁴ in Formula (2). The suitable aspects of R⁴¹ are the same as the suitable aspects of R²¹ to R²⁴.

Y⁴¹ to Y⁴⁵ each independently represent a nitrogen atom or ═CR⁴²—. R⁴² represents a hydrogen atom or a monovalent substituent.

The monovalent substituent represented by R⁴² is not particularly limited, and examples thereof include the substituents listed in the substituent group W described above.

In a case where 2 or more groups among Y⁴¹ to Y⁴⁵ represent ═CR⁴²—, R⁴²'s substituted with adjacent carbon atoms may be linked to each other to form an aromatic or non-aromatic ring.

Furthermore, in a case where Y⁴¹ to Y⁴⁵ represent ═CR⁴²—, the monovalent substituent represented by R⁴² may be linked to R⁴¹ to form an aromatic or non-aromatic ring.

Examples of the compound represented by Formula (4) will be shown below, but the present invention is not limited thereto.

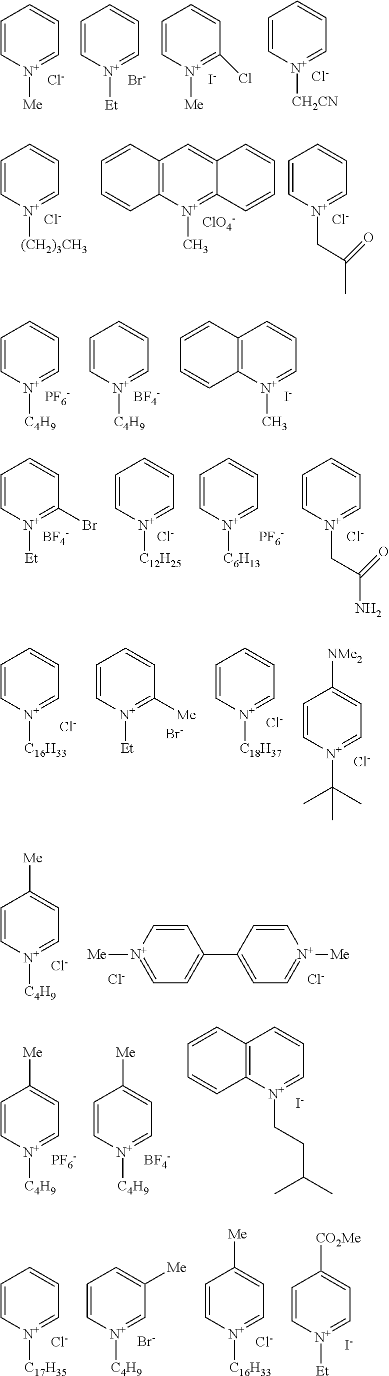

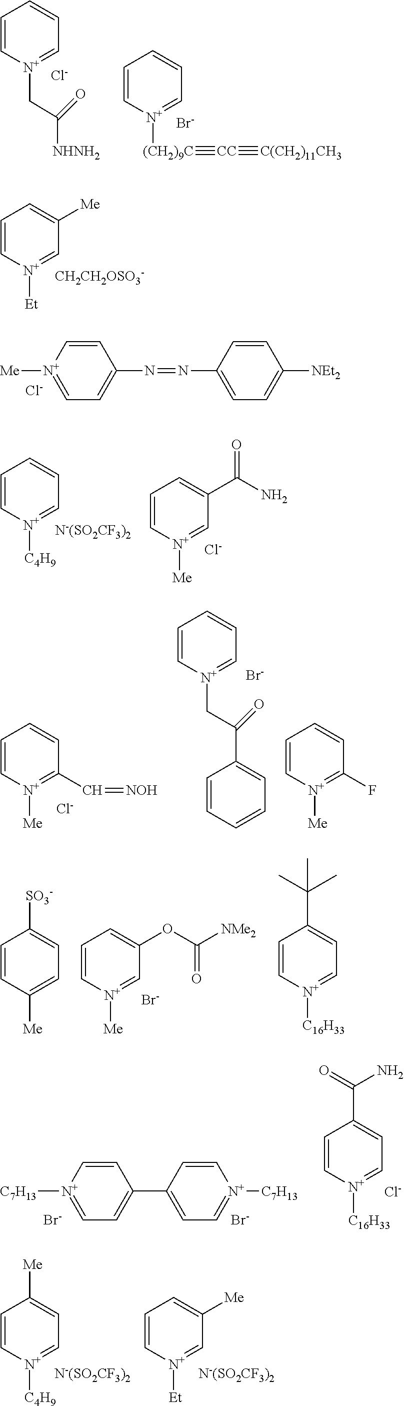

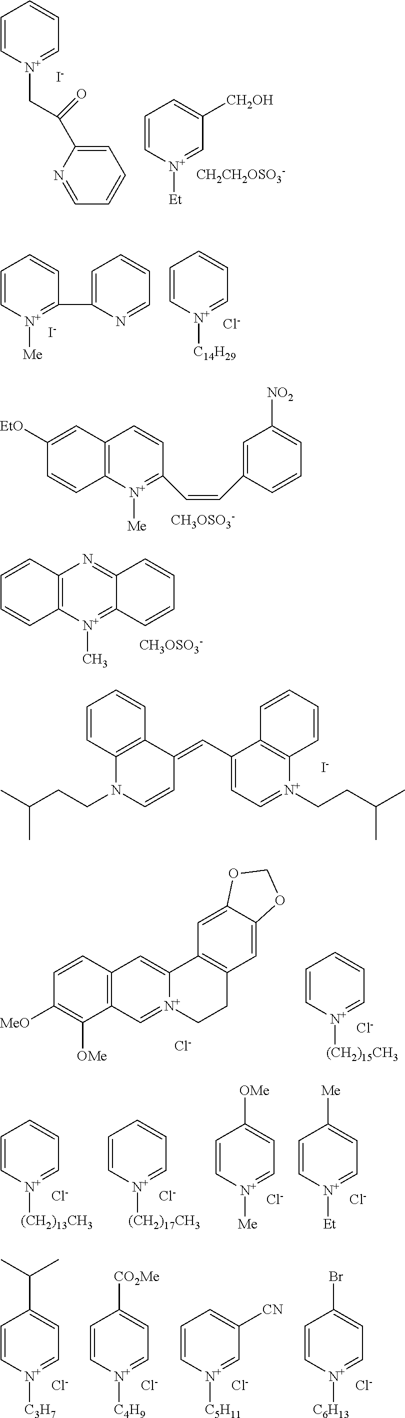

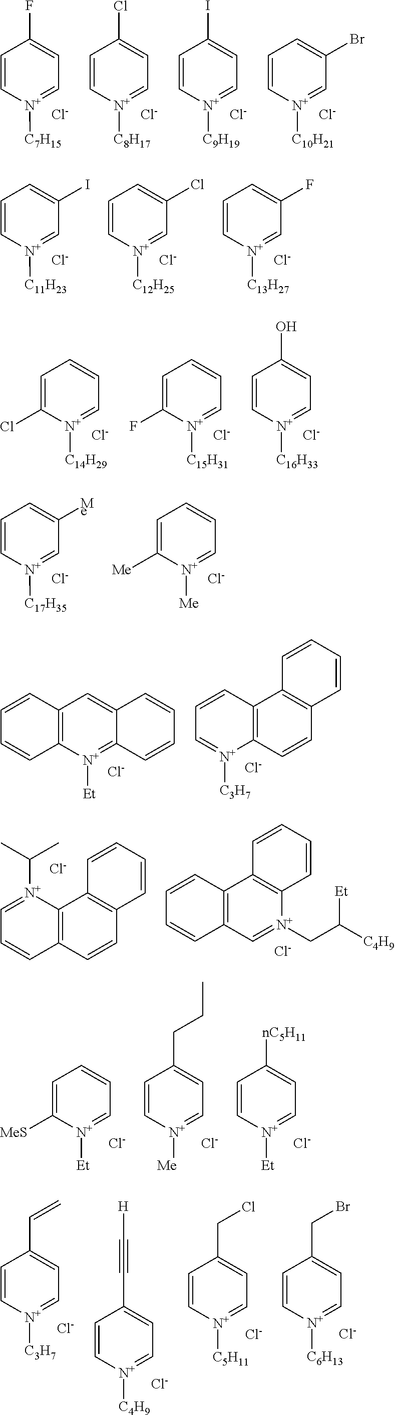

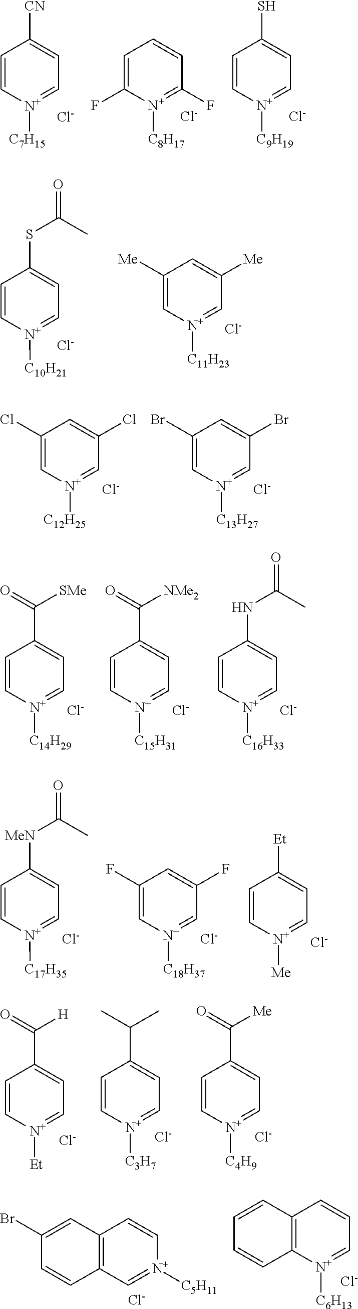

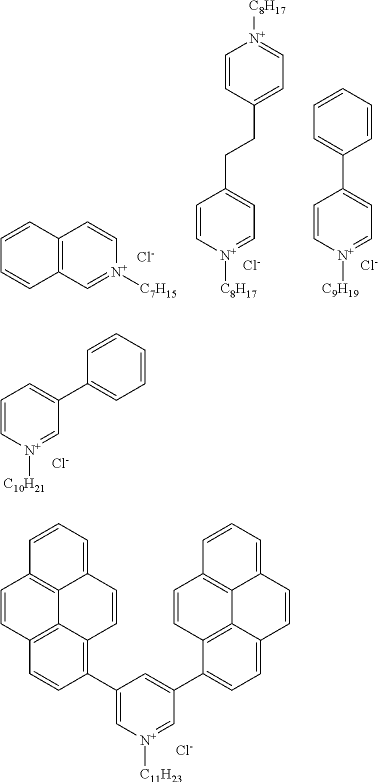

In Formula (5), X⁻ has the same definition as X⁻ in Formula (2). The suitable aspects of X⁻ in Formula (5) are the same as the suitable aspects of X⁻ in Formula (2).

Y⁵¹ to Y⁵³ have the same definitions as Y⁴¹ to Y⁴⁵ in Formula (4). The suitable aspects of Y⁵¹ to Y⁵³ are the same as the suitable aspects of Y⁴¹ to Y⁴⁵.

Y⁵⁴ represents >NR⁵¹, a sulfur atom, or an oxygen atom.

R⁵¹ and R⁵² have the same definitions as R²¹ to R²⁴ in Formula (2). The suitable aspects of R⁵¹ and R⁵² are the same as the suitable aspects of R²¹ to R²⁴.

Examples of the compound represented by Formula (5) will be shown below, but the present invention is not limited thereto.

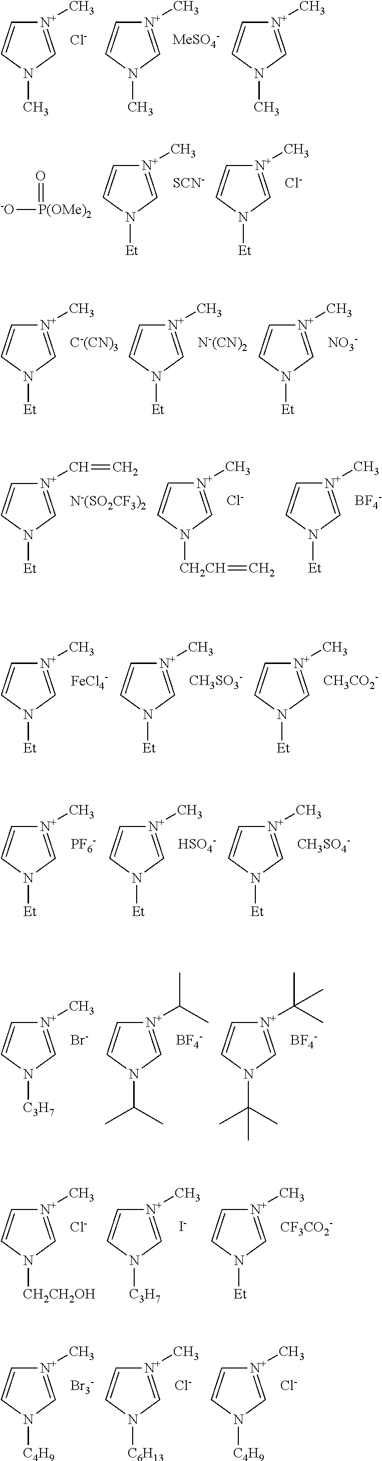

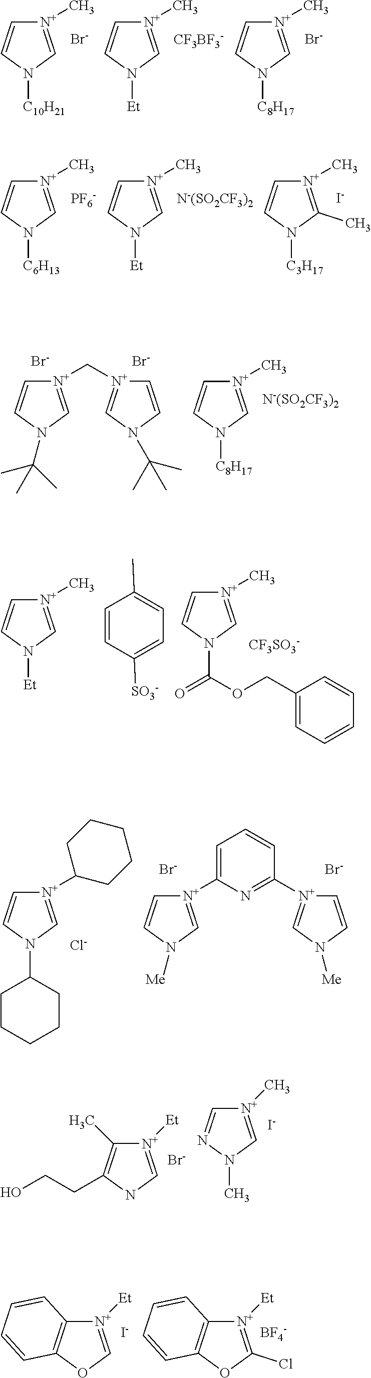

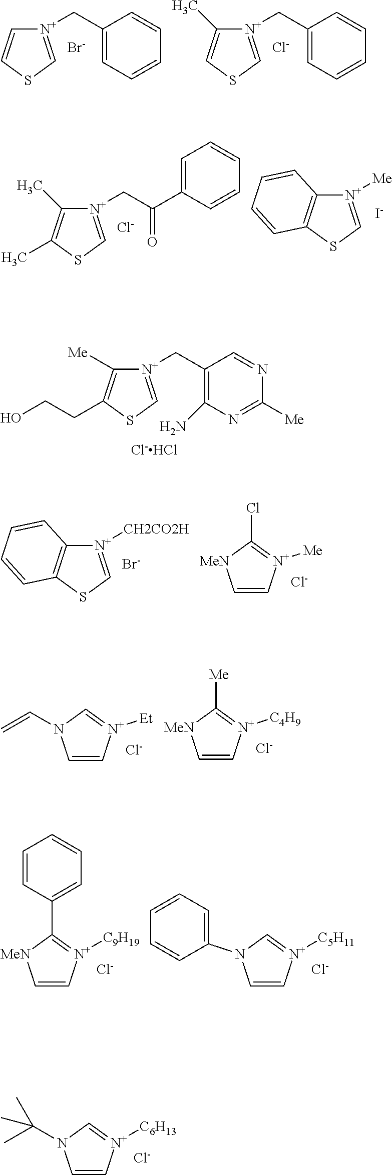

(Metal-Containing Antibacterial Agent)

The metal-containing antibacterial agent is not particularly limited, and known ones can be used.

Examples of the metal include gold, silver, copper, mercury, zinc, iron, lead, bismuth, titanium, tin, and nickel. The form of the metal contained in the metal-containing antibacterial agent is not particularly limited, and examples thereof include metal particles, metal ions, and metal salts (including metal complexes). Particularly, in view of higher antibacterial properties, the metal is preferably gold, silver, or copper.

The metal-containing antibacterial agent may be a metal-supporting carrier including a carrier and the aforementioned metal supported on the carrier.

The type of the carrier is not particularly limited, and known carriers can be used. Examples of the carrier include an inorganic oxide (for example, zeolite (crystalline aluminosilicate), silica gel, silicate such as clay mineral, glass (including water-soluble glass), zirconium phosphate, calcium phosphate, and the like), activated carbon, a metal carrier, and an organic metal.

As the metal-containing antibacterial agent, in view of higher antibacterial properties, a silver-containing antibacterial agent is preferable.

Examples of the silver-containing antibacterial agent include a silver salt such as silver nitrate, silver chloride, silver sulfate, silver lactate, or silver acetate; a silver complex such as a silver-ammonia complex, silver-chloro complex, or a silver-thiosulfate complex; silver particles; silver ions; and a silver-supporting carrier in which the aforementioned antibacterial agent is supported on the carrier described above.

(Photocatalyst)

The photocatalyst is not particularly limited as long as it is a substance known to exhibit photocatalytic activity. Examples thereof include TiO₂, SrTiO₂, ZnO, CdS, SnO₂, and WO₃.

(Aldehyde-Based Compound)

The aldehyde-based compound is not particularly limited, and examples thereof include glutaraldehyde, phthalaldehyde, and formalin.

(Iodine-Based Compound)

The iodine-based compound is not particularly limited, and examples thereof include povidone iodine and iodine tincture.

(Biguanide Compound)

The biguanide compound is not particularly limited, and examples thereof include chlorhexidine gluconate, chlorhexidine hydrochloride, and chlorhexidine acetate.

One kind of each of the bactericide, disinfectant, and fungicide may be used singly, or two or more kinds of bactericides, disinfectants, and fungicides may be used in combination.

In a case where the composition according to an embodiment of the present invention contains the bactericide, the disinfectant, and/or the fungicide, the content of the bactericide, the disinfectant, and the fungicide (total content in a case where the composition contains two or more kinds of bactericides, disinfectants, and fungicides) with respect to the total volume of the composition is preferably 10 to 100,000 mg/L, more preferably 100 to 30,000 mg/L, and even more preferably 100 to 15,000 mg/L.

<Surfactant and Emulsifier>

It is preferable that the composition according to an embodiment of the present invention contain a surfactant and/or an emulsifier. In a case where the composition according to an embodiment of the present invention containing a surfactant and/or an emulsifier is used as a wiper which is prepared by impregnating a base fabric with the composition, the wiper hardly leaves unwiped stains and exhibits higher cleaning properties.

The surfactant is not particularly limited, and examples thereof include an ionic surfactant such as an anionic surfactant, a cationic surfactant, and an amphoteric surfactant, a nonionic surfactant, and the like.

Examples of the anionic surfactant include a higher fatty acid salt such as potassium stearate and potassium behenate; alkyl ether carboxylate such as polyoxyethylene (hereinafter abbreviated to “POE”) sodium lauryl ether carboxylate; N-acyl-L-glutamate such as a N-stearoyl-L-glutamate monosodium salt; a higher alkyl sulfuric acid ester salt such as sodium lauryl sulfate and potassium lauryl sulfate; an alkyl ether sulfuric acid ester salt such as POE triethanolamine lauryl sulfate and POE sodium lauryl sulfate; N-acylsarcosinate such as sodium lauroyl sarcosine; higher fatty acid amide sulfonate such as N-myristoyl-N-methyltaurine sodium; alkyl phosphate such as sodium stearyl phosphate; alkyl ether phosphate such as POE sodium oleyl ether phosphate and POE sodium stearyl ether phosphate; sulfosuccinate such as sodium di-2-ethylhexyl sulfosuccinate, sodium monolauroyl monoethanolamide polyoxyethylene sulfosuccinate, and sodium lauryl polypropylene glycol sulfosuccinate; alkylbenzene sulfonate such as sodium linear dodecylbenzene sulfonate, linear dodecylbenzene sulfonate, and dodecyl diphenyl ether disulfonate; cholate such as sodium deoxycholate, sodium lithocholate, and sodium cholate; and higher fatty acid ester sulfate such as hydrogenated coconut oil fatty acid sodium glycerin sulfate.

Examples of the cationic surfactant include an alkyltrimethylammonium salt such as stearyltrimethylammonium chloride and lauryltrimethylammonium chloride; a dialkyldimethylammonium salt such as distearyldimethylammonium chloride; an alkylpyridinium salt such as poly(N,N dimethyl-3,5-methylene piperidinium) chloride and cetylpyridinium chloride; an alkyl quaternary ammonium salt; an alkyldimethylbenzylammonium salt; an alkylisoquinolinium salt; a dialkylmorpholinium salt; POE alkylamine; an alkylamine salt; a polyamine fatty acid derivative; an amyl alcohol fatty acid derivative; benzalkonium chloride; benzethonium chloride; and the like.

Examples of the amphoteric surfactant include betaine laurylamidopropyl acetate; and an alkyl betaine salt such as coconut oil alkyl betaine and palm kernel oil fatty acid amidopropyl betaine.

As the nonionic surfactant, a compound having more than 20 carbon atoms is preferable. Examples thereof include ester-type compounds such as fatty acid ester compounds of mono-, di-, or polyglycerin, propylene glycol fatty acid monoester, sorbitan fatty acid ester, and sucrose fatty acid ester; ether-type compounds such as polyoxyethylene alkyl ether, polyalkylene alkyl ether, and polyoxyethylene polyoxypropylene glycol (such as EMULGEN series manufactured by Kao Corporation); ester ether-type compounds such as fatty acid polyethylene glycol and fatty acid polyoxyethylene sorbitan; and alkanolamide-type compounds such as fatty acid alkanolamide, and the like.

Specific examples of the nonionic surfactant include polyethylene glycol monolauryl ether, polyethylene glycol monostearyl ether, polyethylene glycol monocetyl ether, polyethylene glycol monolauryl ester, polyethylene glycol monostearyl ester, and the like.

The emulsifier is not particularly limited. In the case of a nonionic emulsifier, the number of carbon atoms thereof is preferably greater than 20. Examples of the emulsifier include oleate (examples of forms of the salt include a calcium salt, a sodium salt, and a potassium salt), caprate (examples of forms of the salt include a calcium salt, a sodium salt, and a potassium salt), caprylate (examples of forms of the salt include a calcium salt, a sodium salt, and a potassium salt), laurate (examples of forms of the salt include a calcium salt, a sodium salt, and a potassium salt), gum rosin glycerin ester, starch sodium octenyl succinate, stearyl citrate, monoglyceride citrate, lactic acid and fatty acid ester compounds of glycerin, fatty acid ester compounds of mono-, di-, or polyglycerin, stearate (examples of forms of the salt include a calcium salt, a magnesium salt, an ammonium salt, an aluminum salt, a potassium salt, and a sodium salt), myristate (examples of forms of the salt include a calcium salt, a magnesium salt, an ammonium salt, an aluminum salt, a potassium salt, and a sodium salt), palmitate (examples of forms of salt include a calcium salt, a magnesium salt, an ammonium salt, an aluminum salt, a potassium salt, and a sodium salt), calcium stearoyl lactylate, sodium stearoyl lactate, solbitan fatty acid ester, sodium dioctyl sulfosuccinate, lecithin, lecithin hydroxide, partially hydrolyzed lecithin, sunflower lecithin, enzyme-treated lecithin, propylene glycol fatty acid ester, polyoxyethylene sorbitan monolaurate, polyoxyethylene sorbitan monostearate, polyoxyethylene sorbitan tristearate, polyoxyethylene sorbitan oleate, a quillaia extract, vegetable sterol, sphingolipid, soybean saponin, bile powder, animal sterol, fractionated lecithin, a Yucca foam extract, egg yolk lecithin, tall oil, and rosin glycerin ester.

As the aforementioned surfactant and emulsifier, among the above, from the viewpoint of safety, food additives are preferable, and cholate (examples of forms of the salt include a calcium salt, a sodium salt, and a potassium salt), deoxycholate (examples of forms of the salt include a calcium salt, a sodium salt, and a potassium salt), oleate (examples of forms of the salt include a calcium salt, a sodium salt, and a potassium salt), caprate (examples of forms of the salt include a calcium salt, a sodium salt, and a potassium salt), caprylate (examples of forms of the salt include a calcium salt, a sodium salt, and a potassium salt), laurate (examples of forms of the salt include a calcium salt, a sodium salt, and a potassium salt), gum rosin glycerin ester, starch sodium octenyl succinate, triethyl citrate, stearyl citrate, monoglyceride citrate, lactic acid and fatty acid ester compounds of glycerin, fatty acid ester compounds of mono-, di-, or polyglycerin, sucrose fatty acid ester, stearate (examples of forms of the salt include a calcium salt, a magnesium salt, an ammonium salt, an aluminum salt, a potassium salt, and a sodium salt), myristate (examples of forms of the salt include a calcium salt, a magnesium salt, an ammonium salt, an aluminum salt, a potassium salt, and a sodium salt), palmitate (examples of forms of the salt include a calcium salt, a magnesium salt, an ammonium salt, an aluminum salt, a potassium salt, and a sodium salt), calcium stearoyl lactylate, sodium stearoyl lactate, solbitan fatty acid ester, sodium dioctyl sulfosuccinate, lecithin, lecithin hydroxide, partially hydrolyzed lecithin, sunflower lecithin, enzyme-treated lecithin, propylene glycol fatty acid ester, polyoxyethylene sorbitan monolaurate, polyoxyethylene sorbitan monostearate, polyoxyethylene sorbitan tristearate, polyoxyethylene sorbitan oleate, a quillaia extract, vegetable sterol, sphingolipid, soybean saponin, bile powder, animal sterol, fractionated lecithin, a Yucca foam extract, egg yolk lecithin, tall oil, or rosin glycerin ester is more preferable.

One kind of each of surfactant and emulsifier may be used singly, or two or more kinds of surfactants and emulsifiers may be used in combination.

In a case where the composition according to an embodiment of the present invention contains the surfactant and/or the emulsifier, the content of the surfactant and the emulsifier (total content in a case where the composition contains two or more kinds of surfactants and emulsifiers) with respect to the total volume of the composition is preferably 100 to 20,000 mg/L, more preferably 500 to 20,000 mg/L, and even more preferably 500 to 10,000 mg/L.

<Antioxidant>

It is preferable that the composition according to an embodiment of the present invention contain an antioxidant. In a case where the composition according to an embodiment of the present invention contains an antioxidant, the antiviral activity is further improved.

The antioxidant is not particularly limited. For example, it is possible to use the various antioxidants described in “Theory and Practice of Antioxidants” (Kajimoto, Sanshobo, 1984) and “Handbook of Antioxidants” (Sawatari, Nishino, Tabata, Taiseisha, 1976).

Examples of the antioxidant include ascorbic acid, ascorbic acid derivatives, and salts thereof; erythorbic acid, erythorbic acid derivatives, and salts thereof; compounds having a phenolic hydroxyl group; and amine-based compounds such as phenylenediamine.

Examples of the ascorbic acid, ascorbic acid derivatives, and salts thereof include L-ascorbic acid, sodium L-ascorbate, potassium L-ascorbate, calcium L-ascorbate, L-ascorbic acid phosphoric acid ester, a magnesium salt of L-ascorbic acid phosphoric acid ester, L-ascorbic acid sulfuric acid ester, a disodium salt of L-ascorbic acid sulfuric acid ester, L-ascorbic acid stearic acid ester, L-ascorbic acid 2-glucoside, L-ascorbic acid plamitic acid ester, and L-ascorbyl tetraisopalmitate.

Examples of the erythorbic acid, erythorbic acid derivatives, and salts thereof include erythorbic acid, sodium erythorbate, potassium erythorbate, calcium erythorbate, erythorbic acid phosphoric acid ester, and erythorbic acid sulfuric acid ester.

Examples of the compound having a phenolic hydroxyl group include a polyphenol compound (for example, catechin contained in a tea extract), nordihydroguaiaretic acid (NDGA), a gallic acid ester compound (for example, propyl gallate, butyl gallate, and octyl gallate), dibutylhydroxytoluene (BHT), butylhydroxyanisole (BHA), a carcinonic acid compound (such as a rosemary extract), ferulic acid, vitamin E, and a bisphenol compound.

Examples of the vitamin E include tocopherol (vitamin E) and derivatives thereof, and tocotrienol and derivatives thereof.

mples of the tocopherol and derivatives thereof include dl-α-tocopherol, dl-β-tocopherol, dl-γ-tocopherol, dl-γ-tocopherol, dl-α-tocopherol acetate, dl-α-tocopherol nicotinate, dl-α-tocopherol linoleate, dl-α-tocopherol succinate, and acetates of these.

Examples of the tocotrienol and derivatives thereof include α-tocotrienol, β-tocotrienol, γ-tocotrienol, δ-tocotrienol, and acetates of these.

Examples of the amine-based compound include phenylenediamine, diphenyl-p-phenylenediamine, and 4-amino-p-diphenylamine.

As the aforementioned antioxidant, among the above, from the viewpoint of safety, food additives are preferable, and 4-hexylresorcin, BHT, butylhydroxyanisole, calcium disodium ethylenediaminetetraacetate, L-ascorbic acid, calcium L-ascorbate, L-ascorbic acid stearic acid ester, sodium L-ascorbate, L-ascorbic acid palmitic acid ester, tert-butylhydroquinone, a d-α-tocopherol concentrate, dl-α-tocopherol, anoxomer, isoascorbic acid, erythorbic acid, sodium erythorbate, isopropyl citrate, guaiac oil, Gum guaicum, dilauryl thiodipropionate, thiodipropionic acid, distearyl thiodipropionic acid ester, sodium thiosulfate, nordihydroguaiaretic acid, potassium pyrosulfite, sodium pyrosulfite, ethyl protocatechuate, ferulic acid, propyl gallate, isoamyl gallate, dodecyl gallate, potassium sulfite, sodium sulfite, potassium hydrogen sulfite, sodium hydrogen sulfite, or stannous chloride is more preferable.

One kind of antioxidant may be used singly, or two or more kinds of antioxidants may be used in combination.

In a case where the composition according to an embodiment of the present invention contains an antioxidant, the content of the antioxidant (total content in a case where the composition contains two or more kinds of antioxidants) with respect to the total volume of the composition is preferably 10 to 20,000 mg/L, more preferably 100 to 10,000 mg/L, and even more preferably 100 to 5,000 mg/L.

<Ultraviolet Absorber>

The ultraviolet absorber is not particularly limited, and examples thereof include salicylic acid-based compounds such as homomentyl salicylate, octyl salicylate, and triethanolamine salicylate; p-aminobenzoic acid-based compounds such as p-aminobenzoic acid, ethyl dihydroxypropyl p-aminobenzoate, glyceryl p-aminobenzoate, octyl dimethyl p-aminobenzoate, amyl p-dimethylaminobenzoate, and 2-ethylhexyl p-dimethylaminobenzoate, benzophenone-based compounds such as 4-(2-β-glucopyranosyloxy)propoxy-2-hydroxybenzophenone, dihydroxydimethoxybenzophenone, sodium dihydroxydimethoxybenzophenone disulfonate, 2-hydroxy-4-methoxybenzophenone, hydroxymethoxybenzophenone sulfonic acid and trihydrate thereof, sodium hydroxymethoxybenzophenone sulfonate, 2-hydro xy-4-methoxybenzophenone-5-sulfonic acid, 2,2′-dihydroxy-4-methoxybenzophenone, 2,4-dihydroxybenzophenone, 2,2′,4,4′-tetrahydroxybenzophenone, 2,2′-dihydroxy-4,4′-dimethoxybenzophenone, and 2-hydroxy-4-N-octoxybenzophenone; cinnamic acid-based compounds such as 2-ethylhexyl p-methoxycinnamate (another name; octyl p-methoxycinnamate), glyceryl mono-2-ethylhexano ate di-p-methoxycinnamate, 2,5-diisopropyl methyl cinnamate, 2,4,6-tris [4-(2-ethylhexyloxycarbonyl)anilino]-1,3,5-triazine, methylbis(trimethylsiloxy)silyl isopentyl trimethoxycinnamate, an isopropyl p-methoxycinnamate. diisopropyl cinnamic acid ester mixture, and a p-methoxyhydrocinnamate diethanolamine salt, benzoylmethane-based compounds such as 2-phenyl-benzimidazole-5-sulfonic acid, 4-isopropyl dibenzoylmethane, and 4-tert-butyl-4′-methoxybenzoylmethane; 2-cyano-3,3-diphenylprop-2-enoic acid-2-ethylhexyl ester (another name; octocrylene), 2-ethylhexyl dimethoxybenzylidene dioxoimidazolidine propionate, 1-(3,4-dimethoxyphenyl)-4,4-dimethyl-1,3-pentanedione, cinoxate, methyl-O-aminobenzoate, 2-ethylhexyl-2-cyano-3,3-diphenylacrylate, 3-(4-methylbenzylidene) camphor, octyl triazone, 2-ethylhexyl 4-(3,4-dimethoxyphenylmethylene)-2,5-dioxo-1-imidazolidine propionate, and polymer derivatives and silane derivatives of these.

One kind of ultraviolet absorber may be used singly, or two or more kinds of ultraviolet absorbers may be used in combination.

In a case where the composition according to an embodiment of the present invention contains an ultraviolet absorber, the content of the ultraviolet absorber (total content in a case where the composition contains two or more kinds of ultraviolet absorbers) with respect to the total volume of the composition is preferably 10 to 30,000 mg/L, more preferably 10 to 20,000 mg/L, and even more preferably 10 to 10,000 mg/L.

<Chelating Agent>

It is preferable that the composition according to an embodiment of the present invention contain a chelating agent (excluding the compound included in the aforementioned component A). The chelating agent is a component which prevents impurity metal ions that can be contained in the base fabric for a wiper and the components of the composition from being precipitated in the base fabric as salts such as carbonate and an oxide salt. In a case where the composition according to an embodiment of the present invention contains a chelating agent, the wiper obtained by impregnating a base fabric with the composition according to the embodiment of the present invention containing the chelating agent less causes uneven wiping and has higher cleaning properties.

The chelating agent is not particularly limited as long as it is a known chelating agent (excluding the compound included in the aforementioned component A). Examples of the chelating agent include an aromatic or aliphatic carboxylic acid-based chelating agent, a phosphonic acid-based chelating agent, a phosphoric acid-based chelating agent, a hydroxycarboxylic acid-based chelating agent, a polymer electrolyte (including oligomer electrolyte)-based chelating agent, dimethylglyoxime, thioglycolic acid, phytic acid, glyoxylic acid, and glyoxal acid. Each of these chelating agents may be in the form of a free acid or a salt such as a sodium salt, a potassium salt, or an ammonium salt.

Examples of the aromatic or aliphatic carboxylic acid-based chelating agent include oxalic acid, malonic acid, succinic acid, glutaric acid, adipic acid, pimelic acid, sebacic acid, azelaic acid, itaconic acid, aconitic acid, pyruvic acid, salicylic acid, acetylsalicylic acid, hydroxybenzoic acid, aminobenzoic acid (including anthranilic acid), phthalic acid, fumaric acid, trimellitic acid, gallic acid, hexahydrophthalic acid, and salts of these.

Examples of the phosphonic acid-based chelating agent include iminodimethylphosphonic acid, alkyldiphosphonic acid, 1-hydroxyethane-1,1-diphosphonic acid, and salts of these.

Examples of the phosphoric acid-based chelating agent include orthophosphoric acid, pyrophosphoric acid, triphosphoric acid, and polyphosphoric acid.

Examples of the hydroxycarboxylic acid-based chelating agent include malic acid, citric acid, glycolic acid, gluconic acid, heptonic acid, tartaric acid, lactic acid, and salts of these.

Examples of the polymer electrolyte (including oligomer electrolyte)-based chelating agent include an acrylic acid polymer, a maleic acid anhydride polymer, an α-hydroxyacrylic acid polymer, an itaconic acid polymer, a copolymer consisting of 2 or more kinds of monomers constituting these polymers, and an epoxy succinic acid polymer.

Particularly, from the viewpoint of safety, the chelating agent is preferably a food additive, and more preferably ethylenediaminetetraacetate (even more preferably ethylenediaminetetraacetic acid, calcium disodium ethylenediaminetetraacetate, or disodium ethylenediaminetetraacetate); L-tartrate (even more preferably L-tartaric acid, potassium L-tartrate, or sodium L-tartrate); citrate (even more preferably citric acid, isopropyl citrate, stearyl citrate, triethyl citrate, calcium citrate, monopotassium citrate, or tripotassium citrate); gluconate (even more preferably gluconic acid, calcium gluconate, or sodium gluconate); polyphosphate (even more preferably polyphosphoric acid, ammonium polyphosphate, calcium polyphosphate, potassium polyphosphate, or sodium polyphosphate); metaphosphate (even more preferably metaphosphoric acid, potassium metaphosphate, or sodium metaphosphate); or phosphate (even more preferably phosphoric acid, potassium hydrogen phosphate, sodium hydrogen phosphate, potassium phosphate, or sodium phosphate).

One kind of chelating agent may be used singly, or two or more kinds of chelating agents may be used in combination.

In a case where the composition according to an embodiment of the present invention contains a chelating agent, the content of the chelating agent (total content in a case where the composition contains two or more kinds of chelating agents) with respect to the total volume of the composition is preferably 10 to 30,000 mg/L, more preferably 10 to 20,000 mg/L, and even more preferably 10 to 10,000 mg/L.

<Moisturizer>

The moisturizer is not particularly limited, and examples thereof include deoxyribonucleic acid, mucopolysaccharide, hyaluronic acid, chondroitin sulfate, aloe extract, gelatin, elastin, chitin, chitosan, a hydrolyzed eggshell membrane, polyoxyethylene methyl glucoside, polyoxypropylene methyl glucoside, sodium lactate, urea, sodium pyrrolidone carboxylate, betaine, and whey.

One kind of moisturizer may be used singly, or two or more kinds of the moisturizers may be used in combination.

In a case where the composition according to an embodiment of the present invention contains a moisturizer, the content of the moisturizer (total content in a case where the composition contains two or more kinds of moisturizers) with respect to the total volume of the composition is preferably 10 to 30,000 mg/L, more preferably 10 to 20,000 mg/L, and even more preferably 10 to 10,000 mg/L.

<Thickener and Gelling Agent>

Examples of the thickener and the gelling agent include a maleic acid anhydride.methyl vinyl ether copolymer, a dimethyldiallylammonium chloride.acrylamide copolymer, an acrylamide.acrylic acid.dimethyldialylammonium chloride copolymer, cellulose or derivatives thereof, keratin and collagen or derivatives thereof, calcium alginate, pullulan, agar, tamarind seed polysaccharide, xanthan gum, carrageenan, high methoxyl pectin, low methoxyl pectin, guar gum, gum arabic, oat gum, acacia gum, crystalline cellulose, arabinogalactan, Karaya gum, tragacanth gum, carob bean gum, gum Ghatti, alginic acid and salts thereof (examples of forms of the salts include an ammonium salt, a potassium salt, a calcium salt, and a sodium salt), alginic acid propylene glycol ester, albumin, casein, curdlan, β glucan and β-glucan derivatives, locust bean gum, gellan gum, cassia gum, mannan, tara gum, gum tragacanth, tamarind gum, dextran, polydextrose, α-glucose, ethylhydroxyethyl cellulose, carboxymethyl cellulose and salts thereof (examples of forms of the salts include a calcium salt and a sodium salt), enzymatically decomposed sodium carboxymethyl cellulose, hydroxypropyl cellulose, hydroxypropyl starch, hydroxypropyl methyl cellulose, methyl ethyl cellulose, methyl cellulose, hydroxypropylated epichlorohydrin crosslinked starch, hydroxypropylated phosphoric acid crosslinked starch, amylopectin, hydroxypropylated phosphoric acid crosslinked starch, acetylated adipic acid crosslinked starch, oxidized hydroxypropylated epichlorohydrin crosslinked starch, acetylated phosphoric acid crosslinked starch, acetylated oxidized starch, alkali-treated starch, glycerol crosslinked starch, acid-treated starch, phosphoric acid monoesterified phosphoric acid crosslinked starch, phosphorylated starch, starch acetate, bleached starch, enzyme-treated starch, oxidized starch, sodium starch glycolate, sodium starch succinate, glucomannan, cyclodextrin, dextrin, pullulan, pectin, sodium polyacrylate, Eucheuma, β-1,3-glucan agar, and derivatives of α-glucose.

One kind of each of the thickener and gelling agent may be used singly, or two or more kinds of thickeners and gelling agents may be used in combination.

In a case where the composition according to an embodiment of the present invention contains a thickener and/or a gelling agent, the content of the thickener and the gelling agent (total content in a case where the composition contains two or more kinds of thickeners and gelling agents) with respect to the total volume of the composition is preferably 10 to 30,000 mg/L, more preferably 10 to 20,000 mg/L, and even more preferably 10 to 10,000 mg/L.

<Preservative>

The preservative is not particularly limited. Examples thereof include benzoic acid, sodium benzoate, potassium sorbate, sodium sorbate, sorbic acid, sodium dehydroacetate, hydrogen peroxide, formic acid, ethyl formate, sodium hypochlorite, propionic acid, sodium propionate, calcium propionate, pectin decomposition product, polylysine, phenoxyethanol, thiram, thiabendazole, Imazalil, diphenyl, natamycin, fludioxonil, azoxystrobin, and tea tree oil.

One kind of preservative may be used singly, or two or more kinds of preservatives may be used in combination.

In a case where the composition according to an embodiment of the present invention contains a preservative, the content of the preservative (total content in a case where the composition contains two or more kinds of preservatives) with respect to the total volume of the composition is preferably 10 to 30,000 mg/L, more preferably 10 to 20,000 mg/L, and even more preferably 10 to 10,000 mg/L.

<Fragrance>

The fragrance is not particularly limited. Examples thereof include musk, acacia oil, anis oil, ylang ylang oil, jasmine oil, sweet orange oil, spearmint oil, geranium oil, neroli oil, mint oil, Hinoki oil, fennel oil, peppermint oil, bergamot oil, lime oil, lavender oil, lemon oil, lemongrass oil, rose oil, rosewood oil, anisaldehyde, civetone, muscone, and limonene.

One kind of fragrance may be used singly, or two or more kinds of fragrances may be used in combination.

In a case where the composition according to an embodiment of the present invention contains a fragrance, the content of the fragrance (total content in a case where the composition contains two or more kinds of fragrances) with respect to the total volume of the composition is preferably 10 to 30,000 mg/L, more preferably 10 to 20,000 mg/L, and even more preferably 10 to 10,000 mg/L.

<Coloring Agent>

The coloring agent is not particularly limited. Examples thereof include a krill coloring agent, an orange coloring agent, kaolin, ultramarine, chromium oxide, iron oxide, titanium dioxide, and chlorophyll.

One kind of coloring agent may be used singly, or two or more kinds of coloring agents may be used in combination.

In a case where the composition according to an embodiment of the present invention contains a coloring agent, the content of the coloring agent (total content in a case where the composition contains two or more kinds of coloring agents) with respect to the total volume of the composition is preferably 10 to 30,000 mg/L, more preferably 10 to 20,000 mg/L, and even more preferably 10 to 10,000 mg/L.

[Composition Manufacturing Method]

The composition according to an embodiment of the present invention can be prepared by appropriately mixing together the above essential components and optional components. The order of mixing together the components is not particularly limited.

[Pharmaceutical Form]

The pharmaceutical form of the composition according to an embodiment of the present invention is not particularly limited. For example, the composition may be in the form of a liquid agent, a gel agent, an aerosol spray agent, or a non-aerosol spray agent.

[Use]

The composition according to an embodiment of the present invention acts to inactivate viruses belonging to families such as the family Caliciviridae, the family Orthomyxoviridae, the family Coronaviridae, and the family Herpesviridae. Therefore, it is preferable to use the composition for reducing the activity of the above viruses by causing the composition to act on the viruses. Examples of viruses belonging to the family Caliciviridae include viruses belonging to the genus Norovirus, the genus Sapovirus, the genus Lagovirus, the genus Nebovirus, and the genus Vesivirus. The composition according to an embodiment of the present invention exerts an excellent inactivating effect particularly on viruses belonging to the genus Norovirus and viruses belonging to the genus Vesivirus.

The composition is preferably used as an antiviral composition. Particularly, the composition is preferably used as an anti-norovirus composition. “Antiviral” means that the composition is used for reducing the activity of a virus by acting on the virus.

How to use the composition is not particularly limited. The composition can be applied or preliminarily applied to sites to which viruses are attached or may be attached. The method of applying the composition is not particularly limited. Examples of the method include a method of spraying the composition on the aforementioned sites, a method of wiping the aforementioned sites with a base fabric containing the composition, a method of covering the aforementioned sites with paper and directly sprinkling the composition thereon directly, and a method of washing hands and fingers with the composition as a liquid cleanser.

[Spray]

The spray according to an embodiment of the present invention includes a spray container and the aforementioned composition stored in the spray container. The composition is as described above.

The spray container may be an aerosol spray container or a non-aerosol spray container. As the spray container, a non-aerosol spray container is particularly preferable.

In a case where the spray container is an aerosol spray container, for example, the spray container contains a gas such as a liquid gas or a compressed gas in addition to the composition. Examples of the aerosol spray container include a spray container containing a gas such as liquefied petroleum gas, dimethyl ether, carbon dioxide gas, nitrogen gas, or isopentane.

In a case where the spray container is a non-aerosol spray container, the spray container substantially does not contain a gas such as a liquid gas or a compressed gas and comprises a mechanism for ejecting a liquid stored in the container out of the container in the form of mist or foam. Examples of the non-aerosol spray container include accumulator-type or direct pressure-type spray containers such as a pump-type spray container and a trigger-type spray container.

[Wiper]

The wiper according to an embodiment of the present invention includes a base fabric and the aforementioned composition with which the base fabric is impregnated. The composition is as described above.

The fibers constituting the base fabric are not particularly limited, and examples thereof include a natural fiber, a synthetic fiber, a semi-synthetic fiber, and a regenerated fiber.

As the fibers constituting the base fabric, one kind of fiber may be used singly, or two or more kinds of fibers may be used in combination.

The natural fiber is not particularly limited, and examples thereof include a cellulosic fiber such as a cotton fiber, a linen fiber, and a pulp fiber; wool, and silk.

The synthetic fiber is not particularly limited, and examples thereof include a vinylon fiber; a vinylidene fiber; a polyester fiber such as a polyethylene terephthalate (PET) fiber, polybutylene terephthalate fiber, a polytrimethylene terephthalate fiber, and a copolymerized polyester fiber; a polyolefin fiber such as a polyethylene (PE) fiber and a polypropylene (PP) fiber; a polyamide fiber such as a nylon 6 fiber, a nylon 66 fiber, a nylon 610 fiber, and a nylon 46 fiber; an acrylic fiber such as a polyacrylonitrile fiber; a polyurethane fiber; a polyvinyl chloride fiber; an aramid fiber; a benzoate fiber; a polychlal fiber; a novoloid fiber; and a polyfluoroethylene fiber.

The semi-synthetic fiber is not particularly limited, and examples thereof include an acetate fiber, a triacetate fiber, and a promix fiber.

The regenerated fiber is not particularly limited, and examples thereof include a rayon fiber, a polynosic fiber, a cupra fiber, and a lyocell fiber.

In view of further improving the antiviral activity of the wiper after long-term storage (hereinafter, also described as “storage stability”), the fibers constituting the base fabric preferably contain a synthetic fiber, and more preferably contain one or more kinds of synthetic fibers selected from the group consisting of a polyolefin fiber (preferably a polyethylene fiber or a polypropylene fiber), a polyester fiber (preferably a polyethylene terephthalate fiber), a vinylon fiber, and a nylon fiber.

In the fibers constituting the base fabric, the content of the synthetic fiber with respect to the total mass of the fibers is, for example, equal to or higher than 30% by mass, preferably equal to or higher than 80% by mass, and even more preferably equal to or higher than 95% by mass. The upper limit of the content of the synthetic fiber is, for example, equal to or lower than 100% by mass with respect to the total mass of the fibers.

The fibers constituting the base fabric are preferably synthetic fibers selected from the group consisting of a polyolefin fiber (preferably a polyethylene fiber or a polypropylene fiber), a polyester fiber (preferably a polyethylene terephthalate fiber), and a vinylon fiber, and more preferably at least one kind of synthetic fiber selected from the group consisting of a polyolefin fiber (preferably a polyethylene fiber or a polypropylene fiber) and a polyester fiber (preferably a polyethylene terephthalate fiber).

In the fibers constituting the base fabric, the content of the cellulosic fiber (total content in a case where the base fabric contains two or more kinds of cellulosic fibers) with respect to the total mass of the fibers is preferably equal to or lower than 70% by mass. In view of further improving the storage stability of the wiper, the content of the cellulosic fiber is preferably equal to or lower than 30% by mass. Particularly, in view of further improving the storage stability of the wiper, it is more preferable that the fibers constituting the base fabric substantially do not contain the cellulosic fiber. Herein, “substantially do not contain” means that the content of the cellulosic fiber with respect to the total mass of the fibers is equal to or lower than 5% by mass. The content of the cellulosic fiber is more preferably equal to or lower than 3% by mass, and particularly preferably equal to or lower than 1% by mass. The lower limit is not particularly limited, but is 0% by mass for example.

The cellulosic fiber means a cellulose-containing fiber or a fiber derived from cellulose. Examples of the cellulosic fiber include a pulp fiber, a rayon fiber, a polynosic fiber, a cupra fiber, a lyocell fiber, an acetate fiber, a diacetate fiber, a triacetate fiber, a cotton fiber, and a linen fiber.

The type of the base fabric is not particularly limited, and examples thereof include a woven fabric, a non-woven fabric, and a knit fabric. Among these, a non-woven fabric is preferable.

The basis weight (mass per unit area) of the base fabric is preferably equal to or lower than 100 g/m². In a case where the base fabric is impregnated with the composition according to an embodiment of the present invention, the amount of the composition is preferably equal to or larger than the amount of the base fabric in terms of mass.

[Wiper Manufacturing Method]

The method of impregnating the base fabric with the composition according to an embodiment of the present invention is not particularly limited. For example, a method may be used in which a base fabric wound in the form of a roll is put in a bottle container so that the roll surface comes into contact with the bottom portion of the bottle container, and the composition according to an embodiment of the present invention is added dropwise to the upper roll surface of the base fabric wound in the form of a roll so that the base fabric is impregnated with the composition according to the embodiment of the present invention.

The wiper is preferably used as an antiviral wiper. Particularly, the wiper is preferably used as an anti-norovirus wiper.

### EXAMPLES

Hereinafter, the present invention will be more specifically described based on examples. The materials, the amount and ratio thereof used, how to treat the materials, the treatment procedure, and the like described in the following examples can be appropriately changed as long as the gist of the present invention is maintained. Therefore, the scope of the present invention is not limited to the following specific examples.

A composition was prepared by the following method, and the antiviral activity of the prepared composition was evaluated. Feline calicivirus was used for the evaluation of antiviral activity. Feline calicivirus is widely known to be used to demonstrate the inactivation action of drugs on norovirus.

### Examples 1 to 30 and Comparative Examples 1 to 3

[Preparation of Composition]

<Preparation of Composition of Example 1>

Ethanol (77.0 mL) and 3.6 mL of isopropanol were added to a glass container containing 976.1 g (5 mmol) of N-methyl-D-glucamine, and the N-methyl-D-glucamine was dissolved in the mixed alcohol solution. Then, water and a 1 mol/L aqueous sodium hydroxide solution were added to the glass container so that the total amount of water was 19.4 mL g and the pH of the prepared composition was 11.5, thereby obtaining a composition of Example 1.

The contents of ethanol, isopropanol, and water in the composition of Example 1 were 77.0% by volume, 3.6% by volume, and 19.4% by volume respectively.

(Measurement of pH)

The pH of the composition of Example 1 was measured by the following method.

pH calibration was performed using a pH standard solution, and then the pH was measured using a pH meter (trade name “pH⋅water quality analyzer LAQUA F-72S”, manufactured by HORIBA, Ltd.) and a pH electrode (trade name “6377-10D”, manufactured by HORIBA, Ltd.). A sample solution was adjusted at a solution temperature of 25° C., the electrode was then immersed in the sample solution and left to stand for about 1 to 2 minutes, and a pH value remain stable on the pH meter was read.

<Preparation of Compositions of Examples 2 to 30 and Comparative Examples 1 to 3>

The compositions of Examples 2 to 30 and Comparative Examples 1 to 3 having the formulation and pH shown in Table 1 were prepared based on the method of preparing the composition of Example 1.

The names and structures of compounds used as the component A for preparing the compositions will be shown below. “CAPS” is an abbreviation for “N-cyclohexyl-3-aminopropanesulfonic acid”.

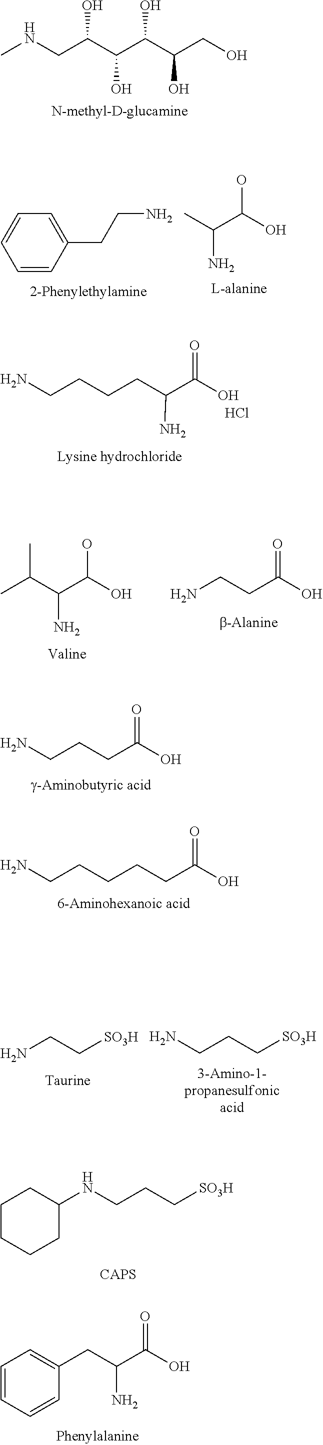

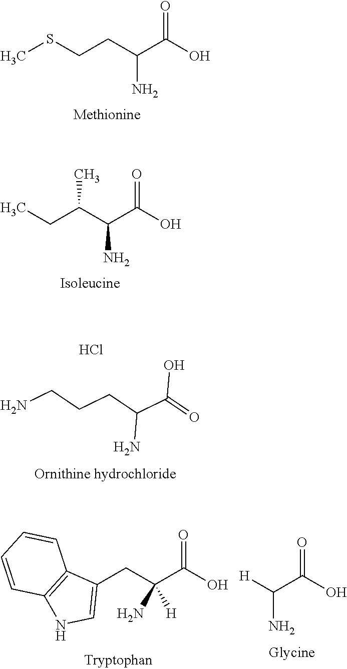

As shown in Table 1, in Examples 21 to 23, the following additives were added to the compositions.

### Example 21

### Urea (Moisturizer)

### Example 22

### 4-Hydroxybenzoic Acid (Chelating Agent)

### Example 23

### Polysorbate 20 (Polyoxyethylene Sorbitan Monolauric Acid Ester, Nonionic Surfactant)

[Evaluation]

By the following method, the antiviral activity of the prepared compositions of Examples 1 to 30 and Comparative Examples 1 to 3 was evaluated.

<Evaluation 1 of Anti-Feline Calicivirus Activity>

A viral solution obtained by culturing Feline calicivirus (ATCC VR-782) in Minimum Essential Media (MEM) was diluted 10-fold with a solution A having the following composition, thereby preparing a virus-containing solution. By using this virus-containing solution, it is possible to evaluate and test the antiviral activity of the composition under conditions closer to the actual environment in which a virus-containing composition (such as feces) is disinfected.

(Composition of solution A)

Liquid medium (trade name “Advanced DMEM/F-12”): 47.6 mL

N-2-Hydroxy-ethyl-piperazine-N′-2-ethanesulfonic acid (HEPES, 1M): 476 μL

Penicillin-streptomycin solution (100-fold concentrated solution) (antibiotic): 476 μL

Fetal Bovine Serum (FBS): 823 μL

Aqueous phosphoric acid solution (8.5% by mass): 605 μL

The virus-containing solution prepared as above was inoculated with the composition of each of the examples or comparative examples at a volume ratio of 1:1, then stirred for 10 seconds, and left to stand at about 25° C. for 1 minute. Thereafter, 0.1 mL of the solution obtained after the inoculation of the viral solution with the composition was collected, added to 9.9 mL of SCDLP medium (Soybean. Casein Digest Agar with Lecithin and Polysorbate 80, supplemented with serum so that the final concentration was 10%), and thoroughly mixed with the medium, thereby obtaining a test solution.

Then, CRFK (Crandell Rees Feline Kidney) cells (established cell line derived from feline kidney, ATCC CCL-94) cultured on an agar medium were inoculated with 0.1 mL of the test solution, and the test solution was allowed to be adsorbed onto the cells at 37° C. for 1 hour. Subsequently, the test solution on the CRFK cells was drained, agar media were layered, and the cells were cultured for 2 to 3 days. After culturing, the number of formed plaques was counted, and the infectious titer was calculated and adopted as “infectious titer of a composition”. Furthermore, the infectious titer was also calculated for a sample prepared in the same manner as described above except that sterilized purified water was used instead of the composition. The calculated titer was adopted as “infectious titer of control”.

The antiviral properties (value of antiviral activity) of the composition were calculated using the following Equation 1, and the calculation result was evaluated based on the following standard. The evaluation results are shown in Table 1.

In addition, the pH of the mixed solution of the viral solution and the composition was measured based on the method for measuring the pH of the composition described above. The pH of the mixed solution (composition after inoculation) of the viral solution and the composition is shown in Table 1.

Value of antiviral activity=A−B Equation 1

A in the above equation represents a common logarithm of the infectious titer of control.

B in the above equation represents a common logarithm of the infectious titer of a composition.

(Evaluation Standard)

“A”: The value of antiviral activity is equal to or higher than 4.0.

“B”: The value of antiviral activity is equal to or higher than 3.0 and less than 4.0.

“C”: The value of antiviral activity is equal to or higher than 2.0 and less than 3.0.

“D”: The value of antiviral activity is less than 2.0.

<Evaluation of Wiping Mark>

By using a spray container, the composition was sprayed three times on the surface of an A3 size (297 mm×420 mm) stainless steel plate. Then, the entire surface of the stainless steel plate was wiped with a dried non-woven fabric so that the sprayed composition spread, and the plate was naturally dried in an environment with a temperature of 23° C. at a humidity of 50% RH. The wiping marks remaining after drying were visually evaluated based on the following standard. The evaluation results are shown in Table 1.

(Evaluation Standard)

“A”: Excellent, no wiping marks are observed.

“B”: There are some wiping marks formed.

“C”: Wiping marks are noticeable.

In Table 1, the column of “Content [mM]” for “Component A” shows the ratio (mM) of the molar amount of the component A to the total volume of the composition. Furthermore, the column of “Content [mg/L]” for “Component A” and “Additive” shows the ratio of the weight (unit: mass mg) of each of the component A and the additive to the total volume (unit: L) of the composition.

Furthermore, in Table 1, each of the columns of “Ethanol [%]”, “IPA [%]”, and “Water [%]” for “Solvent” shows the volume ratio (% by volume) of each of the ethanol, isopropanol, and water to the total volume of the solvent.

TABLE 1

| | Composition | | | | | |
|---|---|---|---|---|---|---|
| | Formulation | | | | | |
| | Component A | | | | Additive | |
| | | Molecular | Content | Content | | Content |
| | Compound name | weight | [mM] | [mg/L] | Compound name | [mg/L] |
| Example 1 | N-methyl-D-glucamine | 195.2 | 50 | 9,761 | — | — |
| Example 2 | 2-Phenylethylamine | 121.2 | 50 | 6,059 | — | — |
| Example3 | L-alanine | 89.1 | 50 | 4,455 | — | — |
| Example 4 | Lysine hydrochloride | 182.7 | 50 | 9,133 | — | — |
| Example 5 | Valine | 117.2 | 50 | 5,858 | — | — |
| Example 6 | β-Alanine | 89.1 | 50 | 4,455 | — | — |
| Example 7 | γ-Aminovaleric acid | 103.1 | 50 | 5,156 | — | — |
| Example 8 | 6-Aminohexanoic acid | 131.2 | 50 | 6,559 | — | — |
| Example 9 | Taurine | 125.2 | 50 | 6,258 | — | — |
| Example 10 | 3-Amino-1-propanesulfonic | 139.2 | 50 | 6,959 | — | — |
| | acid | | | | | |
| Example 11 | CAPS | 221.3 | 50 | 11,066 | — | — |
| Example 12 | Phenylanaline | 165.2 | 50 | 8,260 | — | — |
| Example 13 | Methionine | 149.2 | 50 | 7,461 | — | — |
| Example 14 | Isoleucine | 131.2 | 50 | 6,559 | — | — |
| Example 15 | Ornithine hydrochloride | 168.6 | 50 | 8,431 | — | — |
| Example 16 | Tryptophan | 204.2 | 50 | 10,212 | — | — |
| Example 17 | 6-Aminohexanoic acid | 131.2 | 5 | 656 | — | — |
| Example 18 | 6-Aminohexanoic acid | 131.2 | 10 | 1,312 | — | — |
| Example 19 | 6-Aminohexanoic acid | 131.2 | 90 | 11,806 | — | — |
| Example 20 | 6-Aminohexanoic acid | 131.2 | 400 | 52,469 | — | — |
| Example 21 | 6-Aminohexanoic acid | 131.2 | 66 | 8,657 | Urea | 5,000 |
| Example 22 | 6-Aminohexanoic acid | 131.2 | 66 | 8,657 | 4-Hydroxybenzoic | 5,000 |
| | | | | | acid | |
| Example 23 | 6-Aminohexanoic acid | 131.2 | 66 | 8,657 | Polysorbate 20 | 5,000 |
| Example 24 | 6-Aminohexanoic acid | 131.2 | 90 | 11,806 | — | — |
| Example 25 | 6-Aminohexanoic acid | 131.2 | 90 | 11,806 | — | — |
| Example 26 | 6-Aminohexanoic acid | 131.2 | 90 | 11,806 | — | — |
| Example 27 | 6-Aminohexanoic acid | 131.2 | 90 | 11,806 | — | — |
| Example 28 | 6-Aminohexanoic acid | 131.2 | 90 | 11,806 | — | — |
| Example 29 | 6-Aminohexanoic acid | 131.2 | 2.5 | 328 | — | — |
| Example 30 | 6-Aminohexanoic acid | 131.2 | 400 | 52,469 | | — |
| Comparative | Glycine | 75.1 | 50 | 3,754 | — | — |
| Example 1 | | | | | | |
| Comparative | N/A | — | — | — | — | — |
| Example 2 | | | | | | |
| Comparative | L-alanine | 89.1 | 50 | 4,455 | — | — |
| Example 3 | | | | | | |

TABLE 2

| | Composition | | | | | Evaluation | | |
|---|---|---|---|---|---|---|---|---|
| | Formulation | | | | | pH of | | |
| Continued | Solvent | | | | | composition | | |
| from | Ethanol | IPA | Water | pH | pH of | after | Antiviral | Wiping |
| Table 1 | [%] | [%] | [%] | adjuster | composition | inoculation | activity | mark |
| Example 1 | 77.0 | 3.6 | 19.4 | NaOH | 11.5 | 9.4 | C | B |
| Example 2 | 77.0 | 3.6 | 19.4 | NaOH | 11.5 | 9.4 | B | C |
| Example 3 | 77.0 | 3.6 | 19.4 | NaOH | 11.5 | 8.0 | C | B |
| Example 4 | 77.0 | 3.6 | 19.4 | NaOH | 11.5 | 9.9 | A | B |
| Example 5 | 77.0 | 3.6 | 19.4 | NaOH | 11.5 | 9.0 | C | B |
| Example 6 | 77.0 | 3.6 | 19.4 | NaOH | 11.5 | 9.4 | A | B |
| Example 7 | 77.0 | 3.6 | 19.4 | NaOH | 11.5 | 9.4 | A | B |
| Example 8 | 77.0 | 3.6 | 19.4 | NaOH | 11.5 | 9.6 | A | B |
| Example 9 | 77.0 | 3.6 | 19.4 | NaOH | 11.5 | 8.4 | C | B |
| Example 10 | 77.0 | 3.6 | 19.4 | NaOH | 11.5 | 9.2 | B | B |
| Example 11 | 77.0 | 3.6 | 19.4 | NaOH | 11.5 | 9.6 | B | C |
| Example 12 | 77.0 | 3.6 | 19.4 | NaOH | 11.5 | 8.5 | C | B |
| Example 13 | 77.0 | 3.6 | 19.4 | NaOH | 11.5 | 8.8 | C | B |
| Example 14 | 77.0 | 3.6 | 19.4 | NaOH | 11.5 | 8.4 | C | B |
| Example 15 | 77.0 | 3.6 | 19.4 | NaOH | 11.5 | 9.8 | A | B |
| Example 16 | 77.0 | 3.6 | 19.4 | NaOH | 11.5 | 8.6 | C | B |
| Example 17 | 77.0 | 3.6 | 19.4 | NaOH | 11.5 | 8.0 | C | A |
| Example 18 | 77.0 | 3.6 | 19.4 | NaOH | 11.5 | 8.5 | B | A |
| Example 19 | 77.0 | 3.6 | 19.4 | NaOH | 11.5 | 10.5 | A | B |
| Example 20 | 77.0 | 3.6 | 19.4 | NaOH | 11.5 | 10.5 | A | C |
| Example 21 | 77.0 | 3.6 | 19.4 | NaOH | 11.5 | 10.4 | A | B |
| Example 22 | 77.0 | 3.6 | 19.4 | NaOH | 11.5 | 10.5 | A | B |
| Example 23 | 77.0 | 3.6 | 19.4 | NaOH | 11.5 | 10.4 | A | B |
| Example 24 | 77.0 | 3.6 | 19.4 | NaOH | 10.5 | 9.6 | B | B |
| Example 25 | 77.0 | 3.6 | 19.4 | NaOH | 9.5 | 8.0 | C | B |
| Example 26 | 64.0 | 3.0 | 33.0 | NaOH | 11.5 | 10.5 | B | B |
| Example 27 | 45.0 | 2.1 | 52.9 | NaOH | 11.5 | 10.5 | C | C |
| Example 28 | 64.0 | 3.0 | 33.0 | NaOH | 10.5 | 9.6 | C | C |
| Example 29 | 77.0 | 3.6 | 19.4 | NaOH | 13 | 8.2 | C | A |
| Example 30 | 77.0 | 3.6 | 19.4 | NaOH | 8.5 | 8.0 | C | C |
| Comparative | 77.0 | 3.6 | 19.4 | NaOH | 11.5 | 9.4 | D | B |
| Example 1 | | | | | | | | |
| Comparative | 77.0 | 3.6 | 19.4 | NaOH | 11.5 | 6.0 | D | A |
| Example 2 | | | | | | | | |
| Comparative | 77.0 | 3.6 | 19.4 | — | 7 | 6.0 | D | B |
| Example 3 | | | | | | | | |

From the results in Table 1, it has been confirmed that the compositions of examples exhibit excellent antiviral activity against feline calicivirus.

It has been confirmed that the component A preferably has a carboxylic acid group as the functional group Q because then the antiviral activity is further improved (comparison between Examples 1, 2, and 9 and Example 6, and comparison between Example 7 and Example 10).

In addition, it has been confirmed that the component A preferably has a structure in which an amino group and the functional group Q are bonded through 2 or more atoms (comparison between Examples 3 and 5 and Example 6), and more preferably has a structure in which an amino group and the functional group Q are bonded through 3 or more atoms (comparison between Example 9 and Example 10), because then the antiviral activity is further improved.

It has been confirmed that the content of the component A with respect to the total volume of the composition is preferably equal to or higher than 1,000 mg/L (comparison between Example 17 and Example 18) and more preferably equal to or higher than 3,000 mg/L (comparison between Example 8 and Example 18), because then the antiviral activity is further improved.

In addition, it has been confirmed that the content of the component A with respect to the total volume of the composition is preferably equal to or lower than 30,000 mg/L (comparison between Example 8 and Example 18) and more preferably equal to or lower than 6,000 mg/L, because then the wiping properties are further improved.

It has been confirmed that the content of an alcohol in the composition with respect to the total volume of the solvent is preferably equal to or higher than 50% by volume (comparison between Example 27 and Example 28) and more preferably higher than 70% by volume (comparison between Example 19 and Example 27), because then the antiviral activity is further improved.

It has been confirmed that the pH of the composition is preferably higher than 9.5 (comparison between Example 24 and Example 25) and more preferably higher than 10.5 (comparison between Example 19 and Example 24), because then the antiviral activity is further improved.

<Evaluation 2 of Anti-Feline Calicivirus Activity>

The antiviral activity was evaluated according to the same method as in <Evaluation 1 of anti-feline calicivirus activity>described above, except that the virus-containing solution was inoculated with each of the compositions of Examples 6, 7, and 8 at a volume ratio of 1:1, and then the obtained solution was stirred for 10 seconds and left to stand at about 25° C. for 20 seconds.

As a result, the antiviral properties of the composition of Example 6 were evaluated as C, the antiviral properties of the composition of Example 7 were evaluated as B, and the antiviral properties of the composition of Example 8 were evaluated as A.

From the obtained evaluation results, it has been confirmed that the component A preferably has a structure in which an amino group and the functional group Q are bonded through 3 or more atoms (comparison between Example 6 and Example 7), and more preferably has a structure in which an amino group and the functional group Q are bonded through 4 or more atoms (comparison between Example 7 and Example 8), because then the antiviral activity is further improved.

### Example 31

[Preparation of Wiper]

A wiper was prepared using the liquid agent of Example 21. Specifically, a wiper A was prepared by impregnating a non-woven fabric (base fabric) consisting of a polyolefin fiber (a mixed fiber of a PP fiber and a PE fiber) with the aforementioned liquid agent in an amount of 400% by mass with respect to the total mass of the non-woven fabric, a wiper B was prepared by impregnating a non-woven fabric (base fabric) consisting of 20% by mass of a rayon fiber (corresponding to a cellulosic fiber) and 80% by mass of a polyolefin fiber (a mixed fiber of a PP fiber and a PE fiber) with the aforementioned liquid agent in an amount of 400% by mass with respect to the total mass of the non-woven fabric, and a wiper C was prepared by impregnating a non-woven fabric (base fabric) consisting of 60% by mass of a rayon fiber (corresponding to a cellulosic fiber), 20% by mass of a polyethylene terephthalate (PET) fiber, and 20% by mass of a polyolefin fiber (a mixed fiber of a PP fiber and a PE fiber) with the aforementioned liquid agent in an amount of 400% by mass with respect to the total mass of the non-woven fabric.

Furthermore, a wiper X for preparing a control sample of the wiper A, a wiper Y for preparing a control sample of the wiper B, and a wiper Z for preparing a control sample of the wiper C were prepared by the same method as that described above, except that sterilized purified water was used instead of the liquid agent.

[Various Evaluations]

<Evaluation of Antiviral Activity of Wiper Immediately after Preparation>

Immediately after being prepared, each of the wipers was used for a wiping test with reference to “Method for testing antibacterial effect of wet wipers (revised on Nov. 16, 2015)” established by the Japan Hygiene products Industry Association.

Specifically, according to “Method for testing antibacterial effect of wet wipers (revised on Nov. 16, 2015)” established by the Japan Hygiene products Industry Association, a test carrier (stainless steel plate) was inoculated with a viral solution obtained by culturing feline calicivirus (ATCC VR-782) in Minimum Essential Media (MEM), and the carrier was dried and then wiped a weight wrapped with the wiper A. Thereafter, the test carrier was put in 20 mL of an SCDLP medium, and the residual virus was washed off the test carrier, thereby obtaining a viral solution for preparing a sample. Furthermore, a viral solution for preparing a control sample was obtained in the same manner as described above, except that the wiper X was used instead of the wiper A. Subsequently, CRFK cells cultured on an agar medium were inoculated with 0.1 mL of the viral solution for preparing a sample, and the solution was left to be adsorbed onto the cells at 37° C. for 1 hour. Then, the test solution on the CRFK cells was drained, agar media were layered, and the cells were cultured for 2 to 3 days. After culturing, the number of plaques formed on the agar medium was counted, and the infectious titer was calculated and adopted as “infectious titer of a wiper”. In addition, the infectious titer was also calculated for a control sample prepared in the same manner as described above except that the viral solution for preparing a control sample was used instead of the viral solution for preparing a sample, and the calculated titer was adopted as “infectious titer of a control wiper”.

By using the following Equation 2, the antiviral properties (value of antiviral activity) of the wipers (wipers A, B, and C) were calculated immediately after the wipers were prepared, and the calculation result was evaluated based on the following standard.

Value of antiviral activity=A−B Equation 2

A in the above equation represents a common logarithm of the infectious titer of a control wiper.

B in the above equation represents a common logarithm of the infectious titer of a wiper.

(Evaluation Standard)

“A”: The value of antiviral activity is equal to or higher than 4.0.

“B”: The value of antiviral activity is equal to or higher than 3.0 and less than 4.0.

“C”: The value of antiviral activity is equal to or higher than 2.0 and less than 3.0.

“D”: The value of antiviral activity is less than 2.0.

<Evaluation of Antiviral Activity of Wiper After 6 months of Storage>

Immediately after being prepared, the wipers A, B, and C and the wipers X, Y, and Z for preparing control samples were put in an airtight container and stored in a dark place for 6 months. After the 6 months of storage, the antiviral properties (value of antiviral activity) of the wipers (wipers A, B, and C) were evaluated using the respective wipers by the same method as in <Evaluation of antiviral activity of wiper immediately after preparation>described above.

As a result of the evaluation, it has been confirmed that all of the wipers A, B, and C has antiviral activity evaluated as “A” immediately after preparation, and the composition according to an embodiment of the present invention exhibits excellent antiviral activity even though the composition is used in the form of a wiper.

After the 6 months of storage, the antiviral activity of the wiper A was evaluated as “A”, the antiviral activity of the wiper B was evaluated as “B”, and the antiviral activity of the wiper C was evaluated as “C”.

From the obtained evaluation results, it has been confirmed that the content of the cellulosic fiber in the fibers constituting the base fabric included in the wiper is preferably equal to or lower than 30% by mass (comparison between the antiviral activity evaluation result of the wiper B and the antiviral activity evaluation result of the wiper C) and more preferably equal to or lower than 5% by mass (comparison between the antiviral activity evaluation result of the wiper A and the antiviral activity evaluation result of the wiper B), because then the storage stability is further improved.

What is claimed is:

1. An alkaline composition comprising:
a compound having an amino group and at least one kind of functional group selected from the group consisting of an acidic group, a hydroxyl group, and a phenyl group and having a molecular weight equal to or higher than 85; and
a solvent containing an alcohol.

2. The composition according to claim 1,
wherein the compound is a compound represented by Formula (A) or a salt thereof,
{(Ra)₂N}ₘ-L-(Q)ₙ (A)
in Formula (A), Ra represents a hydrogen atom or a hydrocarbon group having 1 to 10 carbon atoms,
Q represents one kind of functional group selected from the group consisting of an acidic group, a hydroxyl group, and a phenyl group,
m represents an integer of 1 to 3,
n represents an integer of 1 to 5, and
L represents an (m +n)-valent organic group.

3. The composition according to claim 1,
wherein the compound has 1 to 3 amino groups.

4. The composition according to claim 1,
wherein the functional group contains an acidic group.

5. The composition according to claim 1,
wherein the functional group contains a carboxylic acid group.

6. The composition according to claim 1,
wherein the compound has a structure in which an amino group and the functional group are bonded through 2 or more atoms.

7. The composition according to claim 1,
wherein the compound has a structure in which an amino group and the functional group are bonded through 3 or more atoms.

8. The composition according to claim 1,
wherein a content of the compound is 300 to 60,000 mg/L with respect to a total volume of the composition.

9. The composition according to claim 1,
wherein a content of the alcohol is higher than 40% by volume with respect to a total volume of the solvent.

10. The composition according to claim 1, which has a pH equal to or higher than 8.5 and equal to or lower than 14.0.

11. The composition according to claim 1, which is an antiviral composition.

12. The composition according to claim 1, which is an anti-norovirus composition.

13. The composition according to claim 1, which is a liquid agent.

14. The composition according to claim 1, which is a gel agent.

15. A spray comprising:
a spray container; and
the composition according to claim 1 stored in the spray container.

16. A wiper comprising:
a base fabric; and
the composition according to claim 1 with which the base fabric is impregnated.

17. The wiper according to claim 16,
wherein a content of a cellulosic fiber is equal to or lower than 70% by mass with respect to a total mass of fibers constituting the base fabric.

18. The wiper according to claim 16,
wherein a content of the cellulosic fiber is equal to or lower than 30% by mass with respect to a total mass of fibers constituting the base fabric.

19. The wiper according to claim 16,
wherein a content of the cellulosic fiber is equal to or lower than 5% by mass with respect to a total mass of fibers constituting the base fabric.

20. The wiper according to claim 16,
wherein the base fabric contains one or more kinds of synthetic fibers selected from the group consisting of a polyolefin fiber, a polyester fiber, a vinylon fiber, and a nylon fiber.
